(19) United States
(12) Patent Application Publication
(10) Pub. No.: US 2014/0141438 A1
(43) Pub. Date: May 22, 2014

(54) Devices And Method For Positioning Dried Reagent In Microfluidic Devices

(71) Applicant: APPLIED BIOSYSTEMS, LLC, Carlsbad, CA (US)

(72) Inventors: Maengseok Song, Carlsbad, CA (US); Joon Mo Yang, Carlsbad, CA (US); Julie C. Lee, Sunnyvale, CA (US); Nigel P. Beard, San Diego, CA (US); Yuh-Min Chiang, Sunnyvale, CA (US); Roy H. Tan, Carlsbad, CA (US); Carol Schembri, San Mateo, CA (US)

(73) Assignee: APPLIED BIOSYSTEMS, LLC, Carlsbad, CA (US)

(21) Appl. No.: 14/054,383

(22) Filed: Oct. 15, 2013

Related U.S. Application Data
(63) Continuation of application No. 11/422,058, filed on Jun. 2, 2006, now abandoned.

(51) Int. Cl. B01L 3/00; C12Q 1/68

(52) U.S. Cl. CPC B01L 3/502723; C12Q 1/6806
USPC 435/6.12; 422/502; 436/180; 435/287.2

(57) ABSTRACT

A microfluidic device may include a sample distribution network including a plurality of sample chambers configured to be loaded with biological sample for biological testing of the biological sample while in the sample chambers, the biological sample having a meniscus that moves within the sample chambers during loading. The sample distribution network may further include a plurality of inlet channels, each inlet channel being in flow communication with and configured to flow biological sample to a respective sample chamber, and a plurality of outlet channels, each outlet channel being in flow communication and configured to flow biological sample from a respective sample chamber. At least some of the sample chambers may include a physical modification configured to control the movement of the meniscus so as to control bubble formation within the at least some sample chambers. At least some of the sample chambers may include a dried reagent positioned within the at least some sample chambers proximate the inlet channels in flow communication with the at least some sample chambers.

### CROSS-REFERENCE TO RELATED APPLICATION

This application is a continuation of U.S. patent application Ser. No. 11/422,058 filed Jun. 2, 2006, which is incorporated herein by reference.

### FIELD

This disclosure is directed to microfluidic devices and methods and, more particularly, to techniques for filling microfluidic devices so as to hinder the entrapment of gas bubbles.

### INTRODUCTION

Microfluidic devices are used in a wide variety of applications, including, but not limited to, for example, ink jet technology, drug delivery and high-throughput biological assays. In these various applications, various portions within the microfluidic devices may be filled with a substance, such as, for example, a liquid, semi-liquid, or the like. A problem that may be encountered when filling microfluidic devices is the incomplete filling of the portions of the device. Such incomplete filling may be due to the entrapment of residual volumes of gas (e.g., air), thereby forming one or more bubbles, within one or more portions to be filled. It may be desirable to avoid and/or minimize the formation of bubbles within a microfluidic device, as the existence of such bubbles may negatively impact the performance of the device.

For example, in the case of microfluidic devices used for testing and/or analysis of biological samples, such as via polymerase chain reaction (PCR) processes, for example, incomplete filling of portions of the device may negatively impact the reaction efficiency between the sample and, for example, a reagent, and/or the detection of analytes, etc. for which the biological sample is being tested. In some cases, microfluidic devices used for biological testing may rely on optical detection, such as the detection of fluorescence, for example, to determine the presence and/or amount of an analyte of interest. The presence of one or more gas bubbles in the portion of the device at which such optical detection occurs, for example, in a sample chamber of a microcard or other multi-chamber array, may impair the optical detection. Since the level of fluorescence that can be detected increases with the concentration of the various reaction products in a sample chamber, the presence of one or more gas bubbles in the chamber may effectively decrease the concentration of those products, thus decreasing sensitivity of the optical detection. Optical detection may also be impaired due to the presence of a gas bubble within a microcard chamber by altering the path of light entering and/or exiting the chamber. For example, the path of light may be altered due to a lensing effect created by the curvature of the gas bubble surface and/or due to the gas bubble blocking the light.

Also, in the case of biological testing that relies on thermocycling of the sample in a microfluidic device (e.g., a microcard or other multi-chamber array), even a small gas bubble trapped in the device may expand as the device expands.

Further, the presence of a bubble may also impair the reaction efficiency, and thus sensitivity of the device, due to incomplete reactions between, for example, a biological sample, reagent, and/or enzymes being mixed together and used for the biological assay. In some cases, a dried reagent, which may include a nucleic acid target, with or without additional enzymes and the like to support the reaction, may be placed within sample chambers of a microfluidic device. A biological sample, such as a sample containing nucleic acids, for example, may be advanced through the device and into the sample chambers. The entrapment of one or more bubbles in the chamber after filling the chamber with the sample may result in an incomplete mixing of the reagent and the sample, thereby impairing the reaction efficiency and sensitivity of the test.

In some conventional devices, surface treatments, such as, for example, the application of surfactants or plasma processes, have been used on portions of the device which are filled with a substance. Such surface treatments chemically alter the surface and may be used, for example, to increase the hydrophilicity (wettability) of the portions and thereby reduce beading of the substance and subsequent bubble entrapment.

The application of such surface treatments, however, may be difficult to control and may result in nonuniform wettability of the portions being coated. This may lead to nonuniformities in the movement of the substance during filling of the portions and consequent trapping of gas bubbles. Also, the application of these surface treatments may increase the cost and complexity of manufacturing microfluidic devices. Moreover, in some cases, such surface treatments that chemically alter the chamber surface may degrade and/or become ineffective after a time period.

It may be desirable, therefore, to provide a microfluidic device that reduces and/or prevents the formation of bubbles that is relatively simple and inexpensive to manufacture. For example, it may be desirable to provide a microfluidic device that substantially hinders or prevents the formation of gas bubbles that does not rely on surface treatments and/or finishing techniques for which uniformity may be difficult to achieve.

### SUMMARY

Exemplary embodiments according to aspects of the present invention may satisfy one or more of the above-mentioned desirable features set forth above. Other features and advantages will become apparent from the detailed description which follows.

In accordance with various exemplary aspects, the invention may include a microfluidic device in which at least one sample chamber configured to be loaded with a biological sample is modified so as to control the movement of a substance, which may be for example, a liquid, that is supplied to the at least one sample chamber. The at least one sample chamber may be modified to control the movement of a biological sample within the sample chamber and/or to control the movement of a liquid reagent dispensed in the chamber. According to various embodiments, the at least one sample chamber may include a physical modification that is configured to control the movement of the meniscus of a biological sample as it loads the chamber and substantially hinder or prevent the entrapment of a gas bubble within the chamber. Such a physical modification, as used herein, may refer to modifications and/or features of the chamber other than treatments, for example, surface treatments, such as, ozone treatments and/or other surface treatments that chemically alter portions of the chamber so as to reduce and/or prevent bubble formation within a chamber. The physical modifications of the sample chamber in accordance with exemplary aspects of the invention may include a variety of types of features included within the interior of the chamber, as will be explained in further detail below. According to yet further embodiments, the at least one sample chamber may be modified so as to control the location of a dried reagent deposited in liquid form within the chamber. Such a modification may include a modification configured to control the movement of a dispensed liquid reagent to prevent the liquid reagent from spreading to undesired locations within the sample chamber as the reagent dries. Such a modification may be a physical modification and/or a surface modification that alters a hydrophilicity of a portion of the sample chamber.

According to various exemplary embodiments, a microfluidic device may include a sample distribution network including a plurality of sample chambers configured to be loaded with biological sample for biological testing of the biological sample while in the sample chambers, the biological sample having a meniscus that moves within the sample chambers during loading. The sample distribution network may also include a plurality of inlet channels, each inlet channel being in flow communication with and configured to flow biological sample to a respective sample chamber, and a plurality of outlet channels, each outlet channel being in flow communication with and configured to flow biological sample from a respective sample chamber. At least some of the sample chambers may include a physical modification configured to control the movement of the meniscus so as to control bubble formation within the at least some sample chambers.

In accordance with various exemplary embodiments, at least some of the sample chambers of a microfluidic device may include a dried reagent disposed within the at least some sample chambers proximate the inlet channels in flow communication with the at least some sample chambers.

In accordance with yet other exemplary embodiments, a method of filling a microfluidic device may include supplying the microfluidic device with a biological sample, the microfluidic device may include a plurality of sample chambers, a plurality of inlet channels, each inlet channel being in flow communication with and configured to flow biological sample to a respective sample chamber, and a plurality of outlet channels, each outlet channel being in flow communication with and configured to flow biological sample from a respective sample chamber. The method also may include loading the sample chambers with the biological sample, the biological sample having a meniscus that moves within the sample chambers as the biological sample loads the sample chambers. During loading, the method may include controlling the movement of the meniscus via at least one physical modification of at least some of the sample chambers so as to control bubble formation within the at least some sample chambers.

In accordance with yet further various exemplary embodiments, a method of filling a microfluidic device may include supplying the microfluidic device with a biological sample. The microfluidic device may include a plurality of sample chambers, a plurality of inlet channels, each inlet channel being in flow communication with and configured to flow biological sample to a respective sample chamber, and a plurality of outlet channels, each outlet channel being in flow communication with and configured to flow biological sample from a respective sample chamber. A dried reagent may be positioned within at least some of the sample chambers proximate the inlet channels in flow communication with the at least some sample chambers. The method also may include loading the sample chambers with the biological sample.

In the following description, certain aspects and embodiments will become evident. It should be understood that the invention, in its broadest sense, could be practiced without having one or more features of these aspects and embodiments. It should be understood that these aspects and embodiments are merely exemplary and explanatory and are not restrictive of the invention.

### BRIEF DESCRIPTION OF THE DRAWINGS

The drawings of this application illustrate exemplary embodiments of the invention and together with the description, serve to explain certain principles. In the drawings:

FIG. 1 is a plan view of an embodiment of a microfluidic device used for biological testing;

FIGS. 2A-2F show a schematic plan view of exemplary stages of filling of a microfluidic chamber leading to a trapped bubble;

FIG. 3 is a top view of an exemplary embodiment of a microfluidic chamber;

FIG. 3A is a perspective view of an exemplary embodiment of a microfluidic chamber;

FIG. 4 is a top view of another exemplary embodiment of a microfluidic chamber;

FIGS. 5A and 5B are top views of yet further exemplary embodiments of a microfluidic chamber;

FIG. 6A is a top view of yet another exemplary embodiment of a microfluidic chamber;

FIG. 6B is a cross-sectional view of the chamber of FIG. 6A taken from line 6B-6B;

FIG. 7A is a top view of yet another exemplary embodiment of a microfluidic chamber;

FIG. 7B is a cross-sectional view of the chamber of FIG. 7A taken from line 7B-7B;

FIG. 8A is a top view of a yet a further exemplary embodiment of a microfluidic chamber;

FIG. 8B is a partial perspective view of a microfluidic device according to yet another exemplary embodiment;

FIG. 8C is a cross-sectional view of another exemplary embodiment of a microfluidic chamber;

FIG. 8D is a perspective view of yet another exemplary embodiment of a microfluidic chamber;

FIG. 8E is a partial, plan view of a microfluidic device according to yet another exemplary embodiment;

FIGS. 9A-9C are schematic representations of various exemplary embodiments of chambers in microfluidic chips having dried reagent positioned therein;

FIGS. 10A and 10B show photographs of chambers in a microfluidic chip containing centered dried reagent before and after filling, respectively;

FIG. 11A shows photographs of chambers in a microfluidic chip containing centered dried reagent during various stages of filling in which no bubble entrapment occurred;

FIG. 11B shows photographs of chambers in a microfluidic chip containing centered dried reagent during various stages of filling in which bubble entrapment occurred;

FIGS. 12A and 12B show photographs of chambers in a microfluidic chip containing inlet side positioned dried reagent before and after filling, respectively;

FIG. 13A shows photographs of chambers in a microfluidic chip containing inlet side positioned dried reagent during various stages of filling in which no bubble entrapment occurred;

FIG. 13B shows photographs of chambers in a microfluidic chip containing inlet side positioned dried reagent during various stages of filling in which bubble entrapment occurred;

FIGS. 14A and 14B show photographs of chambers in a microfluidic chip containing inlet side positioned dried reagent before and after filling, respectively;

FIG. 15A shows photographs of chambers in a microfluidic chip containing inlet side positioned dried reagent during various stages of filling in which no bubble entrapment occurred;

FIG. 15B shows photographs of chambers in a microfluidic chip containing inlet side positioned dried reagent during various stages of filling in which bubble entrapment occurred;

FIGS. 16A and 16B show photographs of two differing chamber/dried reagent configurations according to exemplary embodiments;

FIGS. 17A-17D schematically depict exemplary embodiments of differing chamber/dried reagent configurations;

FIGS. 18A and 18B show photographs of chambers in a microfluidic chip containing outlet side positioned dried reagent before and after filling, respectively;

FIG. 19 shows photographs of chambers in a microfluidic chip containing outlet side positioned dried reagent during various stages of filling in which bubble entrapment occurred;

FIG. 20 is a chart comparing filling efficiencies calculated for tests of Examples 1-3;

FIG. 21 shows various photographs during filling of a chamber having the configuration of FIG. 16B;

FIG. 22 is a side view of a microfluidic chamber and a branch channel that joins the chamber at a perpendicular angle;

FIG. 23 is a partial plan view of another exemplary embodiment of a microfluidic device used for biological testing;

FIGS. 24A and 24B are partial plan views of yet another exemplary embodiment of a microfluidic device for biological testing; and

FIGS. 25-30 are top views of yet further exemplary embodiments of microfluidic chambers.

FIGS. 25A-30A are cross-sectional views of yet further exemplary embodiments of microfluidic chambers.

### DETAILED DESCRIPTION OF EXEMPLARY EMBODIMENTS

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Wherever possible, the same reference numbers will be used throughout the drawings to refer to the same or like parts.

The section headings used herein are for organizational purposes only, and are not to be construed as limiting the subject matter described. All documents cited in this application, including, but not limited to patents, patent applications, articles, books, and treatises, are expressly incorporated by reference in their entirety for any purpose. In the event that one or more of the incorporated literature and similar materials differs from or contradicts this application, including but not limited to defined terms, term usage, described techniques, or the like, this application controls.

When referring to various directional relationships herein, such as, for example, downward, upward, left, right, top, bottom, etc., such relationships are referred to in the context of the orientation of the drawings, unless otherwise specified. It should be understood, however, that the devices in actuality may be oriented in directions other than those illustrated in the drawings and directional relationships would vary accordingly.

Reference will now be made to various embodiments, examples of which are illustrated in the accompanying drawings. However, it will be understood that these various embodiments are not intended to limit the disclosure. On the contrary, the disclosure is intended to cover alternatives, modifications, and equivalents.

Exemplary aspects of the disclosure provide a microfluidic device configured to be loaded with a biological sample for biological and/or chemical testing. According to various exemplary embodiments, the present invention may provide a device useful for testing one or more fluid samples for the presence, absence, and/or amount of one or more selected analytes. The sample may be a biological sample, for example, an aqueous biological sample, an aqueous solution, a slurry, a gel, a blood sample, a polymerase chain reaction (PCR) master mix, or any other type of sample.

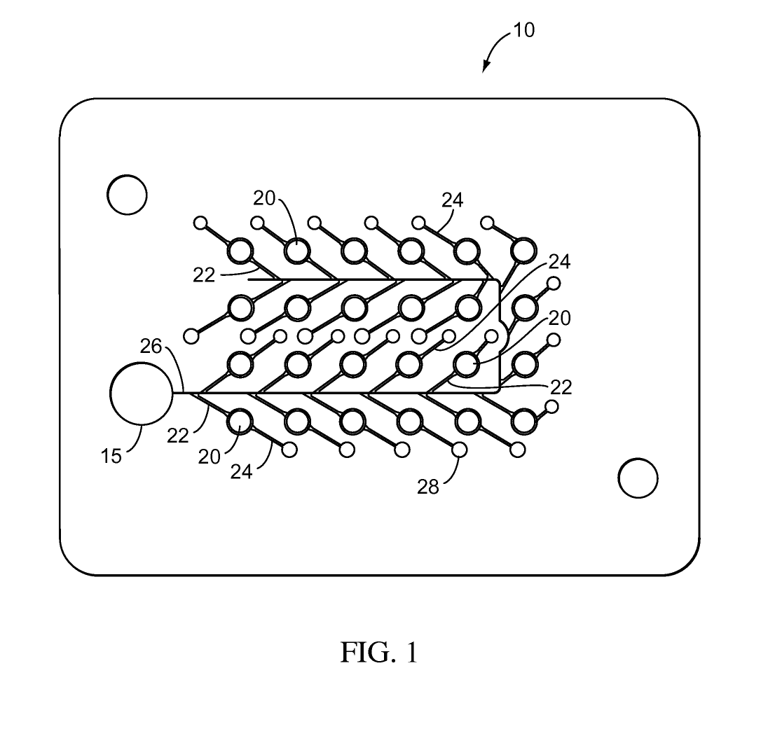

A typical microfluidic device may include a substrate or body structure that has one or more microscale sample-support, manipulation, and/or analysis structures, such as one or more channels, wells, chambers, reservoirs, valves or the like disposed within it. As used herein, “microscale” or “micro” may describe a fluid channel, well, conduit, chamber, reservoir, or other structure configured to move or contain a fluid that has at least one cross-sectional dimension, e.g., width, depth or diameter, of less than about 1000 micrometers. In various embodiments, such structures have at least one cross-sectional dimension of no greater than 750 micrometers, and in some embodiments, from about 1 micrometer to about 500 micrometers (e.g., from about 5 micrometers to about 250 micrometers, or from about 5 micrometers to about 100 micrometers). In one embodiment, the at least one cross-sectional dimension may range from about 50 micrometers to about 150 micrometers. For example, the device shown in FIG. 1 has microchannels with a cross-sectional area 60 μm×150 μm, and microchambers with the diameter of about 1960 μm and the depth of 500 μm.

With respect to chambers, for example, as may be found in a microfluidic card (microcard), chip (microchip) or tray (microtray) used in biological testing, “microscale” or “micro” as used herein, may describe structures configured to hold a small (e.g., micro) volume of fluid, e.g., no greater than about a few microliters. By way of example, the device shown in FIG. 1 may have microchambers with a volume of about 1.35 μL. In various embodiments, such chambers are configured to hold no more than 100 μl, no more than 75 μl, no more than 50 μl, no more than 25 μl, no more than 1 μl. In some embodiments, such chambers can be configured to hold, for example, about 30 μl.

A microfluidic device may be configured in any of a variety of shapes and sizes. In various embodiments, a microfluidic device can be generally rectangular, having a width dimension of no greater than about 15 cm (e.g., about 2, 6, 8 or 10 cm), and a length dimension of no greater than about 30 cm (e.g., about 3, 5, 10, 15 or 20 cm). In other embodiments, a microfluidic device can be generally square shaped. In still further embodiments, the substrate can be generally circular (i.e., disc-shaped), having a diameter of no greater than about 35 cm (e.g., about 7.5, 11.5, or 30.5 cm). The disc can have a central hole formed therein, e.g., to receive a spindle (having a diameter, e.g., of about 1.5 or 2.2 cm). Other shapes and dimensions are contemplated herein, as well.

The present teachings are well suited for microfluidic devices which typically include a system or device having channels, chambers, and/or reservoirs (e.g., a network of chambers connected by channels) for supporting or accommodating very small (micro) volumes of fluids, and in which the channels, chambers, and/or reservoirs have microscale dimensions.

The various sample-containment structures provided within a microfluidic device as set forth herein can take any shape including, but not limited to, a tube, a channel, a micro-fluidic channel, a vial, a cuvette, a capillary, a cube, an etched channel plate, a molded channel plate, an embossed channel plate, or other chamber. Such features can be part of a combination of multiple such structures grouped into a row, an array, an assembly, etc. Multi-chamber arrays within a microfluidic device can include 12, 24, 36, 48, 96, 192, 384, 768, 1536, 3072, 6144, 12,288, 24,576, or more, sample chambers, for example.

In various exemplary aspects, the device may include a substrate defining a sample-distribution network having a main fluid channel for supplying the sample throughout the device, one or more sample chambers (preferably a plurality of such chambers), one or more inlet branch channels providing flow communication between each of the one or more chambers and the main fluid channel, and one or more outlet branch channels in flow communication with the one or more sample chambers. In various exemplary embodiments, the one or more sample chambers may be configured to receive an analyte-specific reagent effective to react with a selected analyte that may be present in a sample that fills the sample chamber. For example, fluorescent probes for amplification of specific nucleic acid targets may be used.

According to various embodiments, the substrate may also have, for each chamber, an optically transparent window through which analyte-specific reaction products can be detected, for example via fluorescence detection mechanisms. The detection mechanism may comprise a non-optical sensor for signal detection.

According to various embodiments, various types of valves can be arranged between the sample chambers and other channels, loading mechanisms, or sample chambers that may be included in or on the device. The valves can be selectively opened and closed to manipulate fluid movement through the device, for example, with the assistance of a centrifugal force or positive displacement. As will be more fully described below and as shown in the drawing figures, the chambers may include a physical modification capable of substantially preventing the entrapment of a gas bubble within the sample chamber during a sample loading procedure. For example, the chamber may include a physical modification configured so as to passively control (e.g., as opposed to actively controlling the pressure or other forces used in flowing the liquid to the chamber) the movement of fluid as it fills the chamber. In other words, the chamber may be modified physically so as to achieve a desired movement of the sample fluid meniscus within the chamber, for example, by achieving a substantially uniform rate and/or manner of movement of the meniscus during loading.

It is contemplated that a variety of techniques may be used to fill the sample chambers and other sample-containment portions of the devices, according to various aspects. For example, filling the various sample-containment portions of the device may occur via centrifuging (e.g., spinning) the device to cause the sample or other liquid to move from, for example, fluid channels into sample chambers. Vacuum also may be used to cause the fluid in the device to move to and/or through various sample-containment portions. According to another exemplary aspect, a positive pressure, applied, for example, via a syringe, pump, or compressor placed in flow communication with a sample-containment structure (e.g., a fluid inlet leading to a main fluid channel) of the device may be used to cause fluid to move throughout the network of sample containment structures in the device to desired portions of the device. In yet another exemplary aspect, capillary forces may be used to move the liquid to desired sample-containment structures of the device. Those having skill in the art would understand how to implement the various techniques discussed above to fill microfluidic devices.

FIG. 1 shows an exemplary embodiment of a microfluidic device 10 used for biological testing. When filling a microfluidic device, such as that exemplified in FIG. 1, the sample fluid may be supplied via an inlet 15 to a main fluid channel 26 from where it travels into a plurality of inlet branch channels 22 leading to a plurality of sample chambers 20. In various exemplary aspects, a syringe, pump, or other positive pressure mechanism may be used to supply the sample to the inlet 15 and fill the microfluidic device 10. The sample fluid fills the sample chambers 20 and exits from outlet branch channels 24 leading from each of the chamber 20. The outlet branch channels 24 are in flow communication with vent chambers 28. According to various exemplary embodiments, the device 10 also may include a film (not shown in FIG. 1), such as, for example, a pressure sensitive adhesive film, laminated to the device so as to cover and seal fluid in the channels and chambers from leaking out of the device. In addition, one or more gas-permeable membranes and/or vent holes provided in a film layer may be provided. Various configurations may be utilized to achieve sealing and gas venting of the device 10, including, for example, the various embodiments disclosed in U.S. application Ser. No. 11/380,327, filed Apr. 26, 2006, having the same assignee, and entitled “Systems and Methods for Multiple Analyte Detection,” the entire disclosure of which is incorporated by reference herein.

A problem that may be encountered during filling of the sample-containment portions of microfluidic devices is the nonuniform advancement of the meniscus formed by the traveling sample through a sample-containment portion. In other words, the meniscus tends to have a start-and-stop motion that results in an uneven motion of the sample front. As a result, one portion of the meniscus may travel at a rate that differs from the rate at which another portion of the meniscus travels. In some cases, the motion of one of the edges of the meniscus (e.g., a portion of the meniscus adjacent one of the lateral walls of the chamber) may lag and/or come to complete stop. This may be caused by an imbalance of the retarding surface forces acting upon the meniscus.

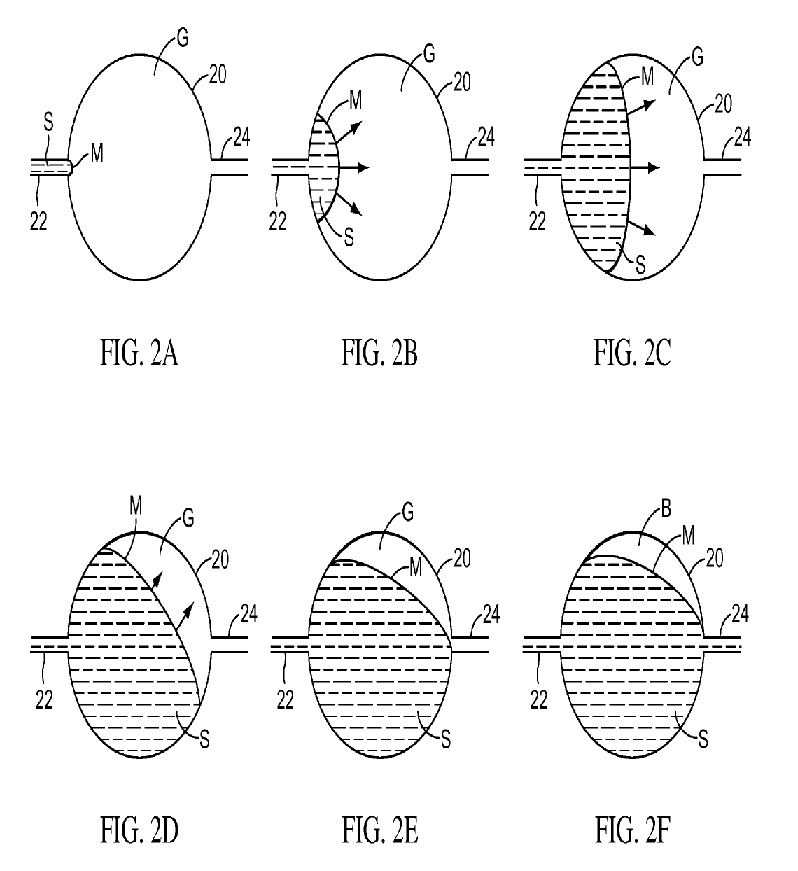

FIGS. 2A-2F schematically depict the advancement of a sample through a sample-containment portion in a microfluidic device leading to an entrapped bubble and therefore an incomplete fill. For example, the sample-containment portion may be in the form of a sample chamber 20 like those shown in FIG. 1. As shown in FIGS. 2A-2F, the sample chamber 20 is in flow communication with two channels. By way of example, the channels may be branch channels 22 and 24 and may provide an inlet to and outlet from the chamber 20, respectively. According to various exemplary aspects, therefore, channel 22 may be an inlet branch channel in flow communication with a main fluid channel like main fluid channel 26 of FIG. 1 (not shown in FIG. 2) so as to receive sample from the main fluid channel to be supplied to the chamber 20. Thus, as shown in FIG. 2A, the sample S may travel via the channel 22 and form a meniscus M that enters the chamber 20 at the inlet opening formed at the junction of the inlet channel 22 with the chamber 20.

FIG. 2B depicts the further progression of the meniscus M and sample S as it begins to load the chamber 20 (namely, in the direction of the arrows). As shown by the shading in FIG. 2B, the sample S fills the channel 22 and a portion of the chamber 20 up to the meniscus M, while the remainder of the chamber 20 (e.g., to the right of the meniscus M shown in FIG. 2B) is filled with a gas (for example, air). FIG. 2C shows the further advancement of the meniscus M and sample S through the chamber 20. In FIGS. 2B and 2C, the movement of the meniscus M is relatively uniform within the chamber 20 such that all portions of the meniscus M appear to be moving in a substantially uniform manner and approaching the outlet opening leading to the outlet branch channel 24 at substantially the same time.

Referring next to FIG. 2D, as the sample S further loads the chamber 20, the meniscus M begins to move unevenly (nonuniformly) in the chamber 20. That is, as depicted by the longer and shorter arrows in the figure, a portion of the meniscus M travels at a faster rate than another portion of the meniscus M. This nonuniformity in the advancement of the meniscus M may cause the portion of the meniscus M that travels faster (the portion proximate the bottom of the chamber 20 in FIG. 2E) to reach the exit channel 24 before the portion of the meniscus M that lags behind (the portion proximate the top of the chamber 20 in FIG. 2E), as depicted in FIG. 2E, for example. When the bottom portion of the meniscus M reaches the outlet channel 24 before the top portion, further sample S that is supplied to the chamber begins to flow through the exit channel 24 and the meniscus M traps gas (e.g., air) within the chamber 20, as shown in FIG. 2F. The result is therefore an incomplete filling of the chamber 20 with the sample S and a gas bubble B trapped in the chamber 20.

As described above, the tendency of the meniscus M to have a nonuniform motion, such as, for example, a stop-and-go motion and/or differing portions moving at differing rates (including, for example, a portion of the meniscus stopping altogether while another portion continues to move), as it moves through the chamber may cause a gas bubble to become trapped within the chamber, as described above with reference to FIGS. 2A-2F. Overall, various movement conditions of the meniscus M, including, but not limited to, differing portions moving at differing rates, one or more portions exhibiting a stop-and-go motion, the complete stopping of one or more portions with other portions continuing to move, and/or a combination of such movements may lead to bubble entrapment in the chamber due to one portion of the meniscus M reaching the outlet channel before the other portion and blocking the outlet channel from letting trapped gas escape. Such movements may occur in any order and at random, and may depend on various factors, such as, for example, filling conditions (e.g., flow rate, pressure), surface conditions (e.g., wettability, surface energy), fluid properties (e.g., viscosity, surface tension), and chamber geometry (shape and dimension, surface roughness, nonuniformities).

Moving the sample within a range of optimal flow rates (e.g., actively controlling the sample flow), for example, by filling the device using a substantially uniform pressure, may make the progress of the sample in the chamber more uniform, thereby decreasing the chances of trapping air. However, as mentioned above, the flow rate may also depend on various other factors, such as, for example, the macro—(e.g., shape) and micro-geometry (e.g., surface roughness) of the chamber, the dimensions of the chamber, the physicochemical surface properties of the chamber (e.g., wettability), and/or properties of the fluid being loaded into the chamber, such as, for example, viscosity, surface tension, density, and/or other fluid properties.

Attempting to produce an optimal flow rate or range of flow rates of the sample during the filling of the chamber in order to control the movement of the meniscus may prove difficult since the flow of the fluid in the chamber, and in particular the motion of the meniscus, may be relatively sensitive to nonuniformities in the finish (e.g., roughness) and wettability of the chamber surface. Thus, techniques for improving the filling of the chamber may include, for example, pre-washing the device to remove contaminants, applying surface treatments to the chamber, and/or modifying the surface roughness of the chambers via suitable manufacturing techniques. In some cases, however, it may be difficult to control the uniformity of the application of such techniques over the area of the chamber surface (e.g., it may be difficult to control such techniques which deal substantially with treating the surface on a micro-level). Thus, in some cases, such techniques may not result in a desired control and/or movement of the meniscus. Also, the application of surface treatments, prewashing, and/or modification to the surface roughness may increase the cost and complexity of manufacturing.

In accordance with various exemplary embodiments, the entrapment of gas bubbles (e.g., air bubbles) during the filling of a microfluidic device may be substantially reduced or eliminated by physically modifying the configuration of one or more sample-containment portions of the device (e.g., such as chambers of the device). In various embodiments, the sample chambers may comprise at least one physical modification (e.g., feature) that is configured to control the movement of the meniscus during loading of the chamber with fluid. For example, such physical modification of the chamber may control the movement of the meniscus of the sample loading the chamber by causing the meniscus to move in a more uniform manner toward the outlet channel. According to various exemplary aspects, this may assist in moving differing portions of the meniscus at substantially the same rate within the chamber, for example, so that substantially the entire sample front can reach the outlet channel (e.g., a plane of the opening of the outlet channel) at substantially the same time.

According to various exemplary embodiments, the chamber may be modified and have a configuration so as to produce a more balanced or uniform distribution of forces (e.g., retarding surface forces, shear forces, and/or pressure forces) that act on the sample as it loads the chamber and/or may create a passive mechanism that acts to stop or slow down the leading portion of the meniscus so that the portion of the meniscus which lags behind has time to advance to the same location as the leading portion. By including one or more features of an appropriate arrangement and configuration in the chamber, an energy/pressure barrier may be encountered by the leading portion of the meniscus so as to increase the surface retarding forces acting on the leading portion and provide the lagging portion of the meniscus a chance to catch up.

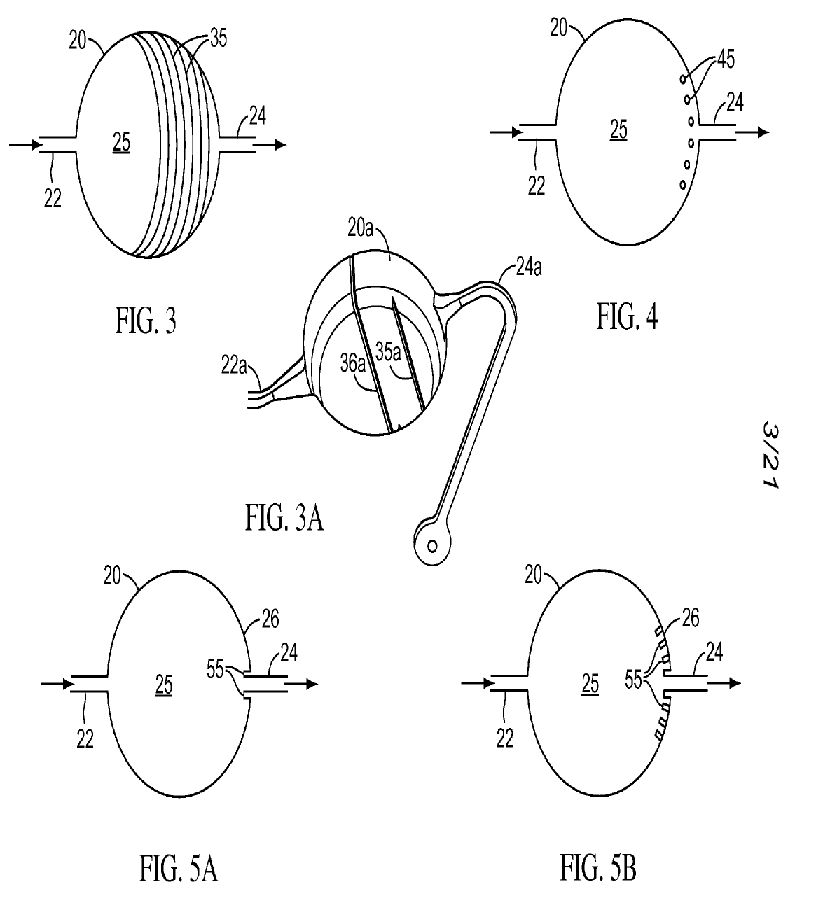

Referring now to FIGS. 3-5B, a plan view of various exemplary embodiments of a chamber are depicted having one or more physical modifications that are configured to provide an energy barrier to slow down or stop the advancement of a leading edge of a meniscus to permit a lagging edge thereof to catch up, thereby controlling the movement of the meniscus as it advances within the chamber to hinder and/or prevent the entrapment of a gas bubble within the chamber.

In FIG. 3, a chamber 20 is shown and is defined by a surface that includes a plurality of grooves 35. More specifically, the plurality of grooves 35 may be provided along a bottom surface portion 25 of the surface defining the chamber 20, as depicted in FIG. 3. In the embodiment of FIG. 3, the grooves 35 are positioned starting approximately midway in the chamber 20 between the inlet channel 22 and outlet channel 24, although other positions for the grooves 35 are also envisioned and may be selected so as to control the motion of the meniscus as has been described herein. In addition or instead of providing grooves on the bottom surface portion 25 of the chamber 20, grooves 35 may be provided on any interior surface portion associated with the chamber 20, including, for example, lateral surface portions (e.g., peripheral surface portions), top surface portions, inlet surface portions and/or outlet surface portions defining the chamber 20. In embodiments wherein grooves are provided on a top surface portion of the chamber, it is envisioned that a plastic material may be bonded to seal the chamber 20, rather than a thin film. In various exemplary embodiments, the grooves may have a depth ranging from 1 micron to about ½ of the chamber depth, for example, on the order of about a few tens of micrometers. The chamber may have a depth of about 500 micrometers, for example.

By providing such grooves 35 on surface portions, for example, bottom surface portion 25, of the chamber 20, if a portion of the meniscus of a fluid sample that is being loaded via vacuum, positive pressure, and/or positive displacement into a chamber 20 that is substantially hydrophobic begins to move faster and lead another portion of the meniscus (as was depicted and described above with reference to FIGS. 2D-2F), that leading portion will encounter the grooves 35 first and experience a retarding surface force that tends to slow or stop the progression of the leading portion. The other, lagging portion of the meniscus may then be able to catch up to the location of the leading portion. Thereafter, the various portions of the meniscus may continue to progress within the chamber 20 in a substantially uniform manner, for example, at substantially the same rate. This may permit the entire sample front to reach the outlet channel 24 (e.g., a plane of the opening leading from the chamber 20 to the outlet channel 24) at substantially the same time to prevent entrapment of a gas bubble.

On the other hand, for filling a hydrophilic chamber 20 either via capillary action or via a combination of capillary action and pressure differential, if differing portions of the meniscus begin to move at differing rates (e.g., nonuniformly) due to either differences in the shear forces acting on the sample and/or competing capillary and pressure forces acting on the sample, the grooves 35 also may be configured so as to provide a balance to the forces (e.g., shear and/or pressure forces) acting on the differing portions of the meniscus, thereby allowing the differing portions of the meniscus to move at the same rate (e.g., allowing one portion to “catch up” to another portion) such that the entire sample front reaches the outlet channel 24 at substantially the same time. In this latter case, therefore, the grooves 35 may act to speed up a portion of the meniscus that is being pulled via capillary forces at a slower rate than another portion of the meniscus.

Although the grooves 35 in FIG. 3 are shown as substantially arc-shaped grooves extending across the entire chamber 20 and substantially perpendicularly to the inlet and outlet channels 22 and 24, it is contemplated that the grooves may have a variety of shapes, sizes, and orientations. By way of nonlimiting example only, the grooves may be substantially straight, diagonal, curved, jagged extending in differing directions within the chamber, continuous, broken (e.g., dashed), have various cross-sectional shapes, and/or any number and/or combinations thereof. Further, in an alternative aspect, instead of grooves, the features can be in the form of reliefs on one or more interior surface portions of the chamber. As with the grooves, such relief features may have a variety of shapes, sizes, configurations and orientations, as discussed above. Further, it is contemplated that grooves and features in relief could be combined together on the chamber surface. Moreover, the spacing between such grooves and/or relief features may vary and or be uniform.

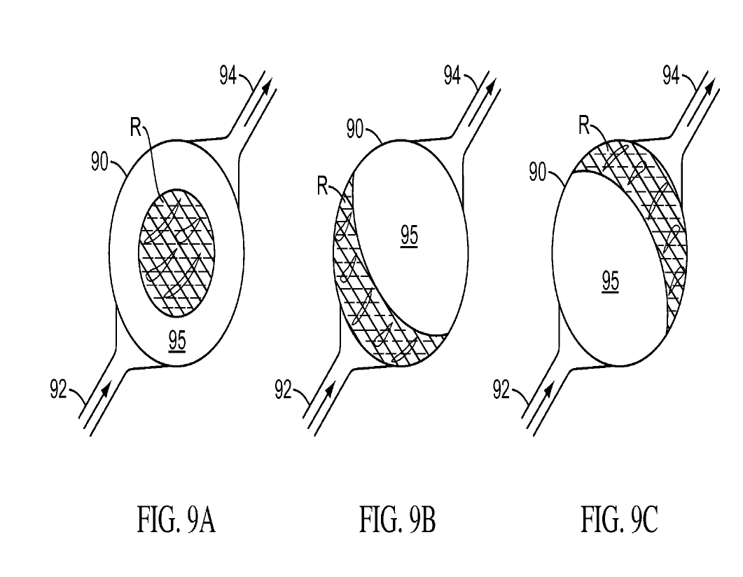

FIG. 3A shows a perspective view of an exemplary embodiment of a sample chamber 20a, taken from an underside of the chamber, that includes relief features in the form of straight ridges 35a and 36a (e.g., like speed bumps) on a bottom surface of the chamber 20a. In the exemplary embodiment of FIG. 3A, two ridges 35a and 36a are provided, with one (36a) being located substantially at a center of the chamber 20a and the other (35a) being located between the center ridge 36a and outlet channel 24a. Aside from acting on the meniscus of the traveling sample so as to control the movement of meniscus to prevent bubble formation, as discussed above, the ridge 36a may provide advantages when spotting dried reagent into the chamber 20a. In accordance with some exemplary embodiments, as discussed in more detail below with reference to FIGS. 9-21 and 23, it may be desirable to spot a liquid reagent in the sample chambers of a microfluidic device and dry the spotted reagent therein. The inventors have found that controlling the position of dried reagent in a chamber may substantially prevent bubble entrapment due to sample loading the chamber. In the exemplary embodiment of FIG. 3A, the ridge 36a positioned at the center of the chamber 20a may act to stop the spread of the liquid reagent past the ridge 36a if the reagent is deposited (e.g., spotted) toward an inlet side of the chamber 20a (e.g., proximate the inlet channel 22a). For reasons that are discussed in more detail below, stopping the liquid reagent from spreading past the ridge 36a positioned at the center of the chamber 20a may be beneficial in controlling the positioning of the dried reagent such that it is located toward the inlet side of the chamber 20a.

FIG. 4 depicts another exemplary embodiment of a chamber 20 having a plurality of projecting members 45 which may, for example, have a pillar-like configuration. As shown in FIG. 4, the projecting members 45 may project upwardly (e.g., vertically) from the bottom surface portion 25 of the surface defining the chamber 20. In various embodiments, the projecting members 45 may be positioned within the chamber 20 proximate the outlet channel 24 and in a substantially symmetrical arrangement with respect to the outlet channel 24, as shown in FIG. 4. In a manner similar to that described above with reference to FIG. 3, the projecting members 45 may act to substantially slow or stop and/or speed up the progression of portions of a meniscus that encounters the projecting members 45 as it advances within the chamber 20 toward the outlet channel 45, depending on the forces in play to move the sample within the chamber and the hydrophobicity or hydrophilicity of the chamber, as described above with reference to the embodiment of FIG. 3.

Referring now to FIGS. 5A and 5B, additional exemplary embodiments of a chamber 20 that includes one or more features configured and arranged to hinder the progression of a leading portion of the meniscus of a sample liquid as it loads the chamber is shown. In the exemplary embodiments of FIGS. 5A and 5B, the surface features include projecting members 55 in the form of teeth. According to various embodiments, and as shown in FIGS. 5A and 5B, the teeth 55 may extend from a lateral surface portion 26 of the surface defining the chamber 20 proximate the outlet channel 24. The teeth 55 may project inwardly toward a center of the chamber 20 and may be positioned on either side of the outlet channel 24 in a substantially symmetrical arrangement. As illustrated in FIGS. 5A and 5B, respectively, one or more teeth 55 may be positioned on each side of the outlet channel 24 (e.g., above and below the channel 24 in FIGS. 5A and 5B). In a manner similar to the grooves 35 and pillars 45, as a leading portion of the meniscus of the sample fluid filling the chamber 20 encounters the teeth 55, the teeth 55 may act to hinder or stop the progression of a leading portion by increasing the surface retarding forces acting on the leading portion. In turn, a lagging portion of the meniscus may be able to catch up to the leading portion, permitting the sample front to reach the outlet channel at substantially the same time.

The use of projecting members, for example, in the form of teeth and/or pillars as set forth in the embodiments of FIGS. 4 and 5 may reduce interference with optical properties on a surface of the chamber (e.g., transparency, etc.). Further, projecting members may be relatively easy to manufacture, for example, by requiring lower dimensional control. Further, according to various embodiments, it may be desirable to position a reagent (e.g., beads of reagent) within the chamber and, in such cases, projecting members may be used to contain the reagent and prevent the reagent from being washed away by the sample through the outlet channel.

According to various exemplary embodiments, the projecting members, whether in the form of teeth or pillars, may range in height such that they extend substantially the entire depth of the chamber 20 or less than the entire depth of the chamber 20. By way of example only, the height of the pillars may range from about 10 microns to the entire depth of the chamber and may have a diameter ranging from about 10 microns to ½ micron. The teeth may have a height ranging from about 10 microns to the entire depth of the chamber, a width ranging from about 10 microns to about ¼ of the chamber perimeter (e.g., circumference), and a length ranging from about 10 microns to about ¼ of the chamber diameter, for example. Moreover, as described for the grooves 35 above, instead of projecting members, the members may be relief features, such as, for example, indentations into the surface portions of the chamber. A combination of such relief features and projecting members also is contemplated.

It also is envisioned that projecting members may be provided on interior surface portions other than the bottom surface portion defining the chamber, such as, for example, lateral, top, inlet and/or outlet surface portions defining the chambers 20. In the case of providing projecting members on a lateral surface portion or top surface portion of the chamber, the projecting members may project from such portions toward a center of the chamber. For example, projecting members may project substantially horizontally from a lateral surface portion defining the chamber. Moreover, it is envisioned that the projecting members may be positioned at various locations in the chamber between the inlet channel 22 and outlet channel 24, and may be aligned or not aligned. The positioning, number, shape, and arrangement of projecting members illustrated in FIGS. 3-5B are exemplary only and not intended to be limiting.

The various surface features depicted in FIGS. 3-5B are exemplary and not intended to be limiting. Those skilled in the art would recognize that the shape, arrangement, dimensions, orientation, spacing, position within the chamber, and number of projecting members, grooves, reliefs or other features may vary and may be selected based on various factors, including, but not limited to, for example, improvement in fluidic performance (e.g., reduction in bubble entrapment), liquid and/or surface physicochemical properties, geometry of the chamber (surface roughness, shape, nonuniformities), filling conditions (flow rates, pressure differentials, centrifugal/centripetal forces due to centrifugal filling), orientation of the device, kinematic or dynamic status of the device, manufacturing constraints, and/or the ability to perform desired optical detection of the chamber. Regarding the ability to perform optical detection, it may be desired to, for example, make various portions of the chamber transparent, opaque, reflective, and/or a combination thereof, create desired refraction patterns within the chamber, create microlenses within the chamber, and/or otherwise control optical detection properties of the chamber. This may also determine the configuration and arrangement, positioning, dimensions, spacing, orientation and number of grooves, reliefs, and/or projecting members. By way of example only, it is envisioned that a single groove, relief feature, or projecting member may be utilized rather than the plurality shown in the figures. Further, aside from grooves, reliefs, or projecting members, it is envisioned that any type of surface feature that alters the forces acting on the portions of the meniscus moving in differing manners (e.g., at differing rates) may be utilized and is considered within the scope of the invention.

Although the description of the embodiments of FIGS. 3-5B discussed the use of pressure and capillary action as the mechanisms for filling the chamber, it is envisioned that the various projecting members, grooves, and reliefs discussed above may also be used in chambers that are filled via centrifuging. For example, the various structures may be used to enable operating of the centrifuge instrument at a lower rpm and/or for a shorter time to achieve chamber filling.

In the embodiments of FIGS. 3-5B, the various features are configured to alter the movement of a portion of the meniscus, for example, a leading portion may be slowed as it approaches the outlet channel 24 of the chamber 20. According to various embodiments, the depth of the chamber may be modified and configured to speed up the movement of the sample fluid toward the sides of the meniscus so as to allow the fluid front proximate a center of the front to lag. This may reduce the tendency of one portion of the meniscus to reach the exit before the other, thereby preventing the entrapment of a gas bubble within the sample-containment portion. Further, expansion ratios may affect filling of a sample chamber due to the sample filling a relatively large volume (e.g., the sample chamber) from a relatively small volume (e.g., inlet channel). To achieve desired filling of the sample chamber, therefore, regions where the inlet and/or outlet channels join the sample chamber may be modified.

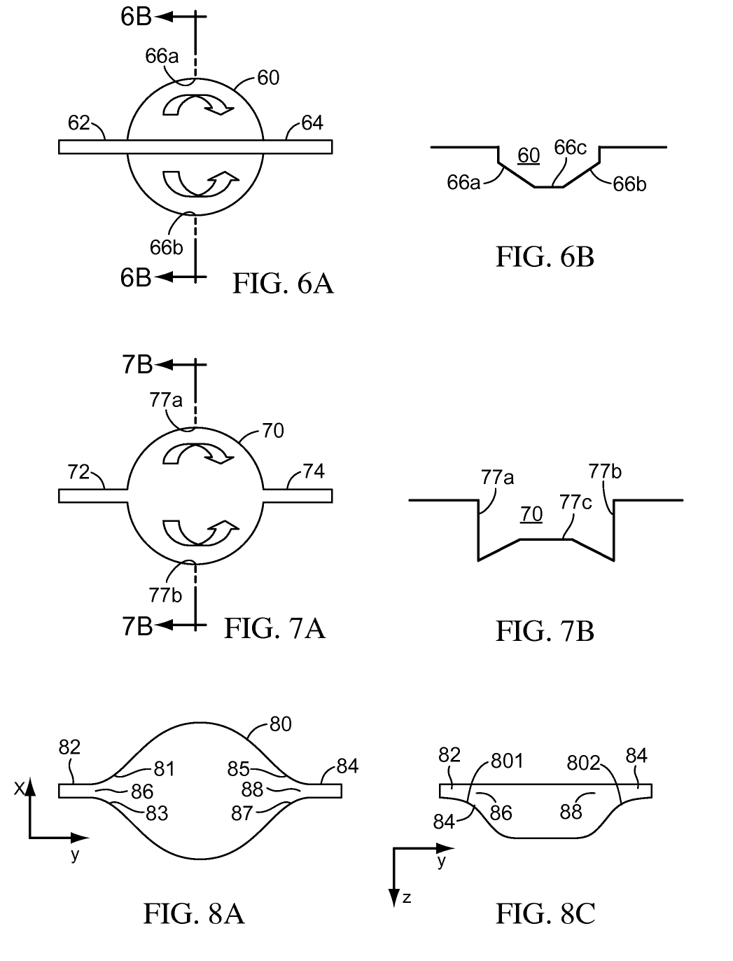

In general, the design of a chamber configured to speed up the movement of the sample fluid toward the sides of the meniscus may depend on the technique used to fill the sample-containment portion. For example, FIG. 6A depicts a plan view of an exemplary embodiment of sample chamber 60 that is configured to increase the rate of movement of the sample fluid located toward the sides (e.g., outer periphery) of the chamber 60. Such an approach may be beneficial when capillary forces are used to fill the chamber 60. The arrows in FIG. 6A are intended to indicate the increased rate of movement of the sample toward the sides (e.g., peripheral surface portions 66a and 66b) of the chamber 60.

In the case of such filling via capillary action, the depth of the chamber 50 proximate the outer periphery of the chamber 60 may be shallower than the center of the chamber 60. In other words, the depth of the chamber 60, as measured from the top, open portion of the chamber to the surface defining the chamber 60 may vary such that the peripheral portions of the chamber 60 are shallower than the center portion of the chamber 60.

FIG. 6B illustrates a cross-sectional view of the chamber 60 taken along line 6B-6B of FIG. 6A. As shown in FIG. 6B, the peripheral surface portions 66a and 66b have a shallower depth within the chamber than the central surface portion 66c. Varying the depth in the manner depicted in FIG. 6B may thus result in a chamber 60 having a substantially bowl-like shape, as opposed to, for example, a substantially cylindrical shape. Similarly, in various embodiments, the portion of the chamber where the lateral surface portions meet the bottom surface may be rounded rather, for example as depicted in FIG. 3A, rather than meeting at a sharp, 90 degree angle.

Providing a chamber 60 wherein the depth of the surface within the chamber 60 is shallower proximate the periphery of the chamber 60, as exemplified in FIG. 6B, for example, may increase the capillary forces acting on the sample fluid, and thus the meniscus, proximate those portions. This may create a siphoning effect during loading of the chamber 60 with the sample fluid, which in turn may permit the outer edges of the meniscus of the sample liquid (e.g., those portions of the meniscus proximate the peripheral surface portions of the chamber 60) to progress faster and allow the center portion of the meniscus to lag such that one side of the meniscus does not reach the outlet channel 64 before another side.

In a case where pressure is used to drive a filling of chamber 60 with the sample fluid, such as via a pump, syringe, centrifuging, or vacuum, it may be desirable to reduce the flow resistance proximate a periphery of the chamber 60. By reducing the flow resistance around the periphery of the chamber 60, the rate of flow of the sample as it fills the chamber 60 may be increased, as was described above with reference to FIG. 6A. Thus, for example, FIG. 7A depicts a plan view of an exemplary embodiment of sample chamber 70, similar to the chambers 20 of FIG. 1, that is configured to increase the rate of movement of the sample fluid located toward the sides (e.g., outer periphery) of the chamber 70, as indicated by the arrows in the figure.

As illustrated in FIG. 7B, to achieve a decrease in flow resistance (and increase in rate of progression of the sample) proximate a periphery of the chamber 70, portions 77a and 77b proximate the edge of the chamber 70 (e.g., the peripheral surface portions of the chamber 70) have a greater depth than a portion 77c located proximate the center of the chamber 70. The greater depth of the surface portions 77a and 77b within the chamber 70 permits the portions of the chamber 70 proximate those surface portions (e.g., the periphery of the chamber 70) to fill faster, thus causing an increase in the rate of movement of the meniscus along the periphery of the sample chamber (e.g., the upper and lower portions shown in FIG. 7A) and a lag in the rate of movement of the central portion of the meniscus. As described above with reference to FIG. 6, this tends to reduce the tendency of one side of the meniscus to reach the outlet channel 74 before the other side, thereby hindering or preventing the entrapment of a bubble within the chamber 70.

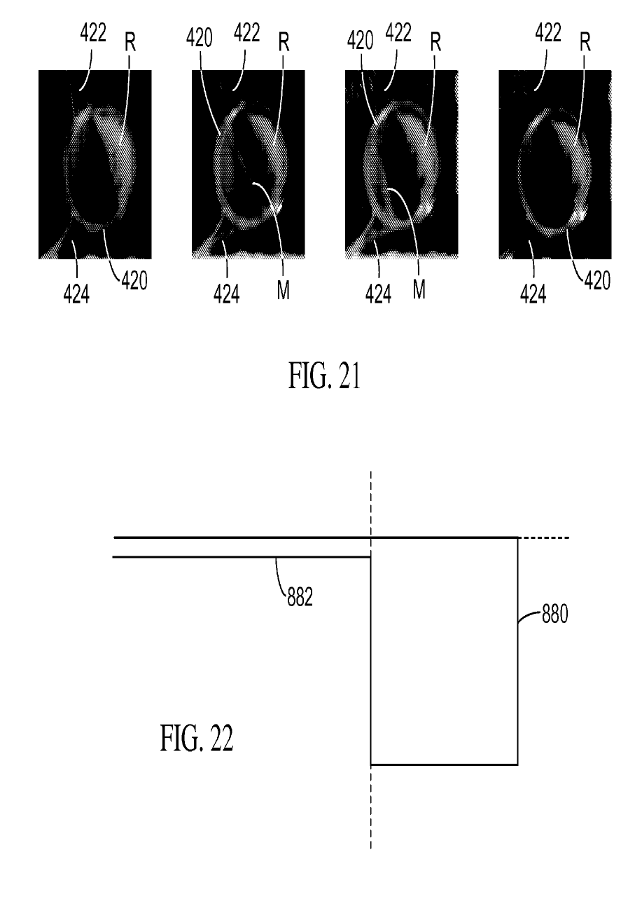

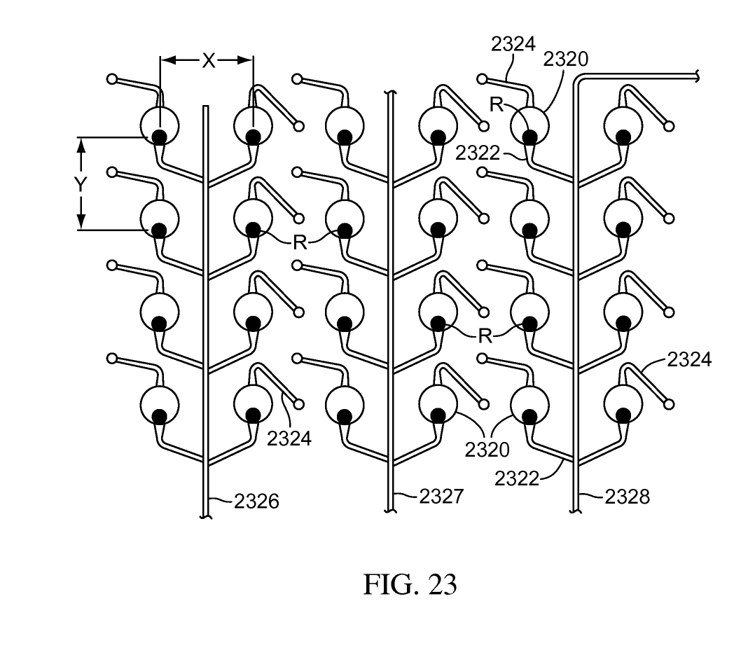

In yet further various embodiments, the transition between the inlet channel and/or the outlet channel and the chamber may be modified, for example, so as to increase the size of the openings that lead to the inlet and/or the outlet channels. In a conventional chamber structure of a microcard, the sample chamber has a substantially cylindrical configuration and the inlet and outlet channels join the chamber at a substantially orthogonal angle, for example, as schematically depicted in FIG. 22 (with reference numeral 880 indicating the chamber, reference numeral 882 representing the inlet channel, and the outlet channel not being shown). In other words, the interior surface portions of the chamber that join the interior surface portions defining the lumen of the inlet and/or the outlet channel intersect each other orthogonally. With such a configuration, the openings leading to the inlet and outlet channels are relatively narrow.

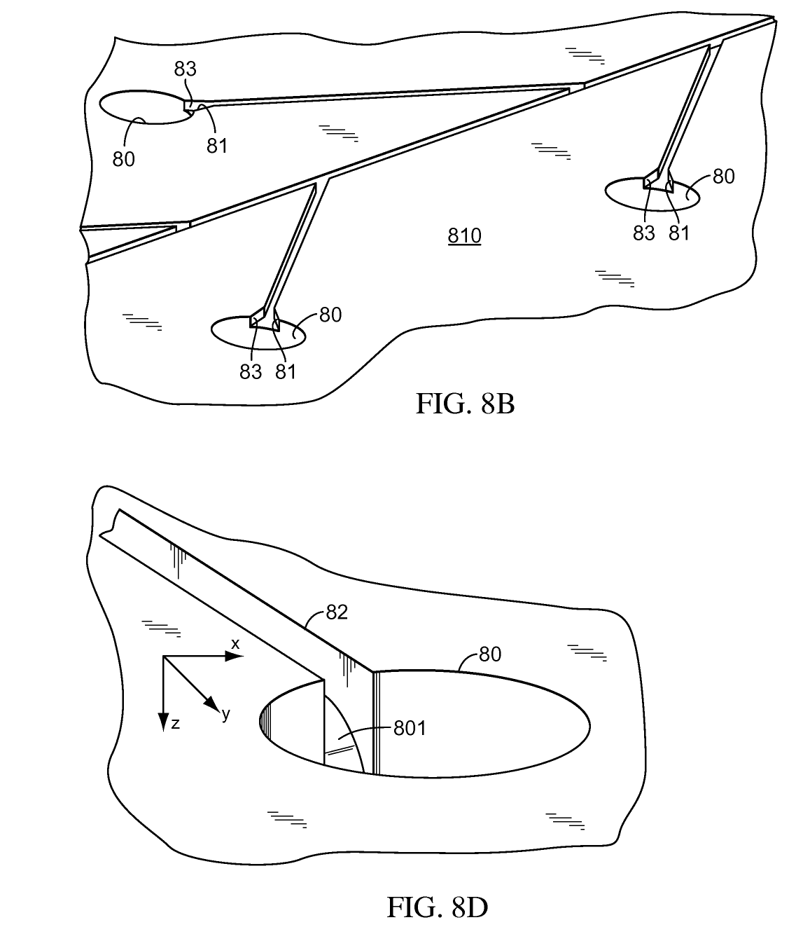

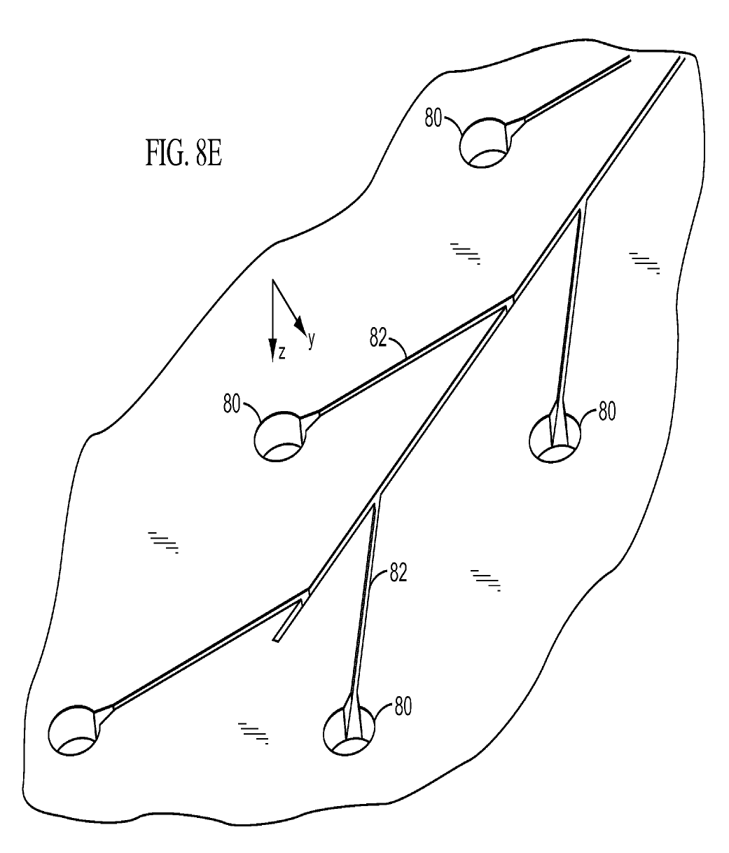

FIG. 8A depicts a top view of an exemplary embodiment of a sample chamber 80 in which portions of the surface defining the chamber 80 that are proximate openings 86 and 88 leading to the inlet and outlet channels 82 and 84, respectively, are configured to provide a smooth transition to the channels 82 and 84. That is, as illustrated in FIG. 8A, the surface portions 81, 83, 85, and 87 are non-orthogonal to the interior surface portions of the lumens defined by the inlet and outlet channels 82 and 84. Providing a non-orthogonal junction between the channels 82 and 84 and the surface defining the chamber 80 may increase the size of the openings leading to the channels 82 and 84. With such an increased opening at the outlet channel 84, the tendency for the meniscus to block the opening and outlet channel 84 is reduced, as is the tendency to trap a bubble within the chamber 80.

Further, providing a smooth transition at the inlet channel 82 (e.g. radius), may enhance the uniformity of the pressure field, thereby promoting uniformity in the movement of the sample meniscus through the chamber. For example, the expansion ratio may be decreased so as to improve filling of the sample chamber.

FIG. 8B is a perspective view of a portion of a microfluidic device 810 comprising sample chambers 80 in flow communication with inlet channels 82. As depicted in FIG. 8B, the interior surface portions 81 and 83 meet the interior surface portions of the lumens defined by the inlet channels 82 at a nonperpendicular angle. In other words, the portions 81 and 83 fan outwardly relative to the longitudinal axis of the channel 82 proximate upstream of the portions 81 and 83. By way of example only, the portions 81 and 83 may fan outwardly at an angle ranging from about 30 degrees to about 60 degrees, for example, at about 30 degrees. Although FIG. 8B does not illustrate outlet channels, it should be understood that such outlet channels could be provided and have transition portions similar to those depicted in FIG. 8B and described above. Moreover, it should be understood that one or both of the inlet and outlet channels leading to a sample chamber may join the chamber at a nonperpendicular angle, as discussed with reference to FIGS. 8A and 8B.

The inlet and/or outlet regions of the chamber may thus be modified from the typical orthogonal intersection of the inlet and outlet channels with the chamber by, for example, including a radius, an angle, or a higher-order polynomial shape where the interior surface portions of the inlet and/or outlet channels meet the interior surface portions of the chamber. It should be understood that the transitional profiles of the inlet and outlet regions (e.g., the surfaces where the inlet and outlet channels meet the surface defining the chamber) may be the same or may differ from each other.

Further, in an alternative exemplary aspect, interior surface portions other than those shown in FIG. 8A may include a smooth transition. FIG. 8C depicts a side view of an exemplary embodiment of the chamber 80 wherein the chamber 80 includes interior surface portions 801 and 802 that meet the respective interior surface portions defining the lumens of the inlet and outlet channels 82 and 84 at a nonorthogonal angle. In other words, as depicted in FIG. 8C, the interior surface portions 801 and 802 substantially form a radius that offers a smooth transition in the z-direction between the inlet and outlet channels 82 and 84, thereby increasing the size of the inlet opening 86 and outlet opening 88. As discussed above, it should be understood that one or both of the inlet and outlet regions may include the smooth transition depicted in FIG. 8C. FIG. 8D depicts a perspective view of a chamber 80 having an inlet channel 82 joining the chamber 80 at a nonorthogonal angle in the z-direction, as described above with reference to FIG. 8C. That is, the interior surface portion 801 joins an interior surface portion of the lumen defining the inlet channel 82 at a nonperpendicular angle. FIG. 8D does not show an outlet channel in flow communication with the chamber 80. However, it should be understood that such an outlet channel may be provided and may or may not join the chamber at a perpendicular angle.

The inlet and outlet channels may include differing transitions, for example, differing radii sizes and/or differing shapes. Moreover, according to various exemplary embodiments, one or both of the inlet and the outlet may provide the transitions shown in FIGS. 8A and 8B (e.g., smooth transitions in the x-direction) in combination with those shown in FIGS. 8C and 8D (e.g., smooth transitions in the z-direction). With reference to FIG. 8E, for example, a smooth transition is provided in both the x-direction and z-direction between the inlet channels 82 and the chambers 80 shown in that figure. Alternatively, the transitions of FIGS. 8A and 8B and of FIGS. 8C and 8D may be used independently of each other and need not be combined.

According to yet further exemplary embodiments, the overall shape of the sample chamber may be modified so as to assist in avoiding bubble entrapment during filling. For example, the shape of the sample chamber may be changed from having a substantially circular cross-sectional configuration to a more elongated shape, such as, for example, an oval-like (e.g., elliptical) cross-sectional configuration. Narrowing the dimensions of the chamber in the direction substantially perpendicular to the direction of flow of sample through the chamber (in other words elongating the chamber substantially in the direction of the sample flow), while substantially maintaining the volume of the chamber, the meniscus of the sample may move through the chamber in a substantially uniform manner such that the entire meniscus reaches an outlet of the chamber at substantially the same time.

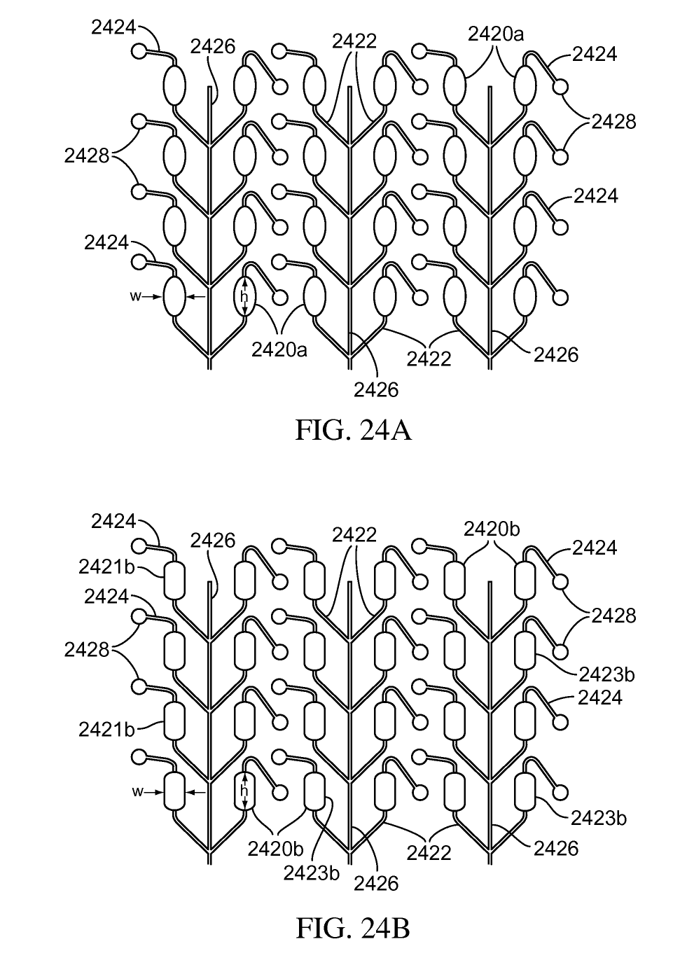

FIGS. 24A and 24B show partial plan views of exemplary embodiments of microfluidic devices that include main fluid channels 2425 in flow communication with a plurality of inlet branch channels 2422 leading respectively to a plurality of sample chambers 2420a or 2420b, and a plurality of outlet branch channels 2424 connecting each sample chamber 2420a and 2420b to a vent chamber 2428. Thus, in FIG. 24, sample is supplied toward a bottom of each of the main fluid channels 2425 and flows in a direction toward the inlet channels 2422, into the sample chambers 2420a and 2420b, out of the outlet channels 2424, and into the vent chambers 2428. In the exemplary embodiments of FIGS. 24A and 24B, the sample chambers 2420a and 2420b are modified from the substantially circular shapes depicted for example in FIG. 1 to substantially elongated shapes in the direction of sample flow through the chambers. In FIG. 24A, the sample chambers 2420a have substantially oval shapes and in FIG. 24B, the sample chambers 2420b have a substantially oval shape with flattened lateral wall portions 2421b and 2423b. The flattened lateral wall portions may facilitate machining and/or molding of the chambers 2420b. As discussed above, the sample chambers 2420a and 2420b may have a volume that is substantially the same as the volume of a chamber having a substantially circular configuration. Thus, in accordance with the teachings herein, the volume of each chamber 2420a and 2420b may be about 1.35 μL and the depth may be, for example, about 500 μm. In various other exemplary embodiments, such chambers are configured to hold no more than 100 μl, no more than 75 μl, no more than 50 μl, no more than 25 μl, no more than 1 μl. In some embodiments, such chambers can be configured to hold, for example, about 30 μl. According to various exemplary embodiments, the short axis dimension w (e.g., diameter) of the sample chambers 2420a and 2420b may range from about 1.0 mm to about 1.8 mm, and the long axis dimension h (e.g., diameter) may range from about 2.1 mm to about 3.2 mm. By way of example, the short axis dimension w (e.g., diameter) may be about 1.32 mm and the long axis dimension h (e.g., diameter) may be about 2.56 mm. It should be understood, that the sample chambers may have elongated shapes other than those depicted in FIGS. 24A and 24B, including, but not limited to, a substantially rectangular shape, for example.

The arrangement of the various channels and chambers depicted in FIGS. 24A and 24B is exemplary only and other arrangements in accordance with the teachings herein are contemplated as within the scope of the invention. However, the arrangement shown in FIGS. 24A and 24B, which may provide for a substantially even pitch between the inlet side regions of the chambers 2420a and 2420b of each device, may provide advantages when providing the sample chambers 2420a and 2420b with a dried reagent, as will become apparent from the description of the exemplary embodiment of FIG. 23 below.

For a variety of applications of microfluidic devices, including, for example, when using microfluidic devices for biological testing, dried reagents may be placed (e.g., “spotted”) into sample-containment portions of the device so that when the devices are filled with a sample to be analyzed, the sample and the reagents may mix as the sample loads a sample-containment portion. Providing dried reagents may improve the stability of various components at room temperature, including, for example, proteins such as DNA/RNA polymerases. As used herein, the term “dried reagent” or variations thereof means liquid reagent where liquid has been at least partially removed by processes where the liquid reagent is, for example, lyophilized, freeze dried, vacuum dried, or gas dried, for example, air dried, nitrogen dried, or dried by any other inert gas (not reacting or interacting with any reagent to be dried in the liquid reagent), where the gas can be at ambient temperature, heated, or cooled, for example, ambient air and/or the gas can be at ambient pressure or compressed, for example, compressed nitrogen, or forced, for example, forced air, by any means including, but not limited to, fan or blower. Further, portability of the microfluidic device and sensitivity of PCR may be additional advantages since dried reagents can be relatively easily stored and a sample solution containing PCR targets is not diluted when mixed with dried PCR reagents. For at least some of these various reasons, dried reagents are deposited in the chambers of microfluidic devices, such as, for example, microfluidic chips, trays, or cards.

Typically, liquid reagents are dispensed in the center of the chambers of a microfluidic device, such as that depicted in FIG. 1, for example, and dried (e.g., lyophilized). FIG. 9A schematically depicts an exemplary embodiment of a microfluidic sample chamber 90 having a dried reagent R deposited on a bottom surface 95 defining the chamber 90. The reagent R is deposited substantially at the center of the chamber 90. It has been found that with a centered positioning of the dried reagent R, variable fill results occur when loading the chamber 90 with a sample fluid, such as, for example an aqueous nucleic acid solution via inlet channel 92. In some cases, the fill efficiency was found to be worse in the presence of the centered dried reagent R than in the case where no dried reagent is present in the chamber. The filling efficiency, FEc, may be calculated as FEc (%)=100*(WFc/Wcd), where Wcd is the number of chambers having centered dried reagent per microfluidic chip and WFc is the number of such chambers with no bubble formation after filling the chambers having centered dried reagent.

Based on 100 tests performed for a microfluidic device such as that shown in FIG. 1, the average fill efficiency was about 85% for 24 chambers with no reagent in the chambers. As will be explained further below, the average fill efficiency for tests in which centered dried reagent was placed in the chamber, as depicted schematically in FIG. 9A, was about 47.6%.

To improve filling efficiency and substantially hinder or prevent the entrapment of bubbles within a chamber containing dried reagent, it has been found, in accordance with the invention, that the chamber may be physically modified via selective positioning of dried reagent within the chamber so as to achieve a desired movement of the meniscus of the sample fluid as it fills the chamber. More specifically, the inventors have discovered that the meniscus may propagate through the chamber in a more uniform manner based on the position of the dried reagent within the chamber.

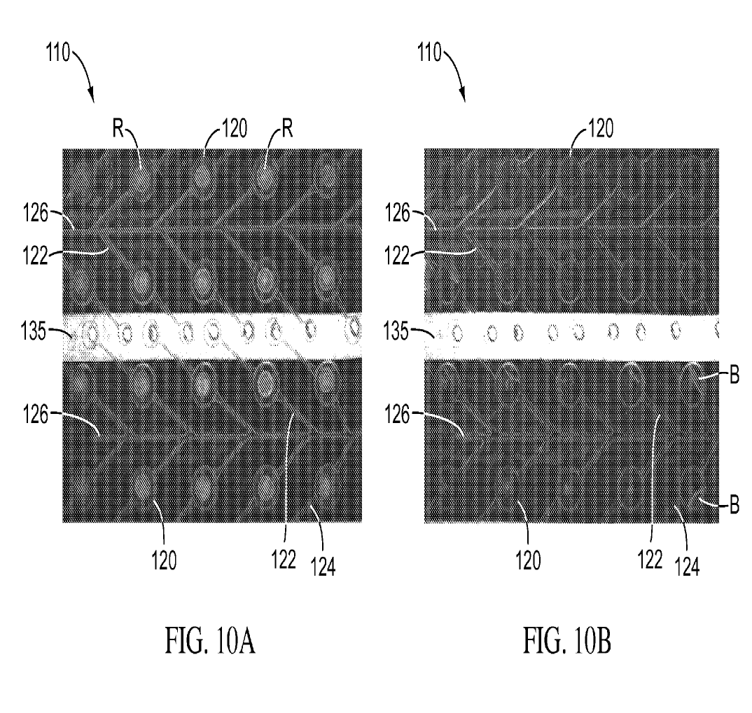

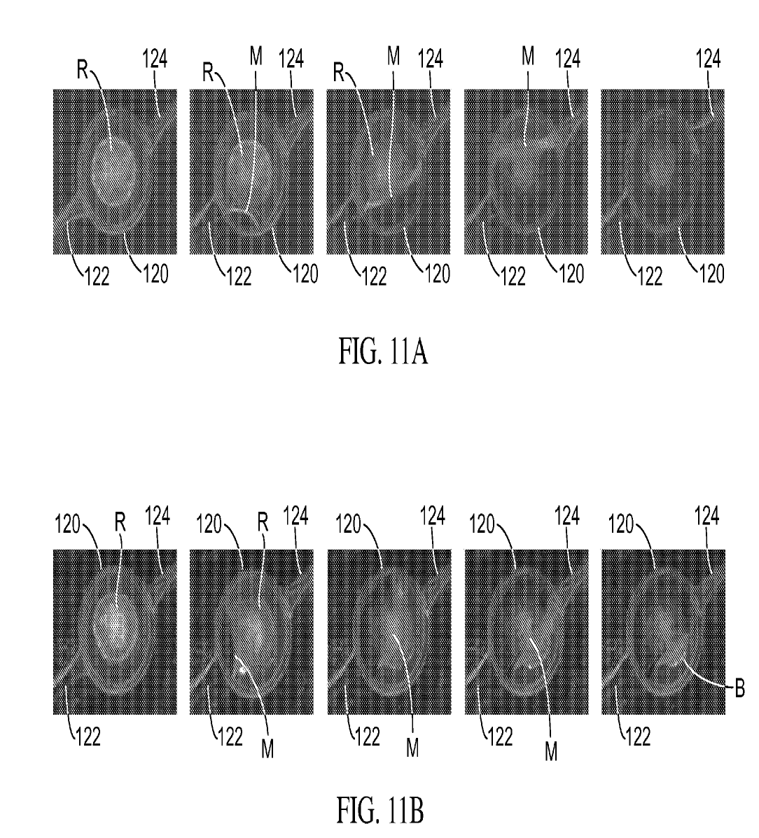

To compare the effect of the position of the dried reagents within the chamber on the filling performance, liquid reagents were dispensed in the center, proximate the inlet channel, and proximate the outlet channel of the chambers of microfluidic chips having a structure similar to that schematically depicted in FIG. 1. Each of the chambers had a volume of about 1.35 μL. The reagents were positioned in the chambers, which were formed in a substrate comprising a cyclic olefin polymer (COP) substrate, via an automatic dispenser and were dried on the chips. The chips containing the dried reagents were laminated with a double-sided pressure sensitive adhesive (PSA) film (not shown in FIG. 1) to seal the chambers and channels formed in the COP substrate. The film included vent holes that align with the vent chambers 28 depicted in FIG. 1, and a plurality of gas-permeable, liquid-impermeable membrane strips were placed on the side of the PSA film opposite the COP substrate and over each row of vent holes and vent chambers 28 (one such membrane strip can be seen in each of FIGS. 10A, 10B, 12A, 12B, 14A, 14B, 18A, and 18B). For further details on the laminated double-sided PSA film, vent holes, and membrane strips used, reference is made to U.S. application Ser. No. 11/380,327, filed on Apr. 26, 2006, assigned to the same assignee as this application, and entitled “Systems and Methods for Multiple Analyte Detection,” the entire disclosure of which is incorporated by reference herein.

The chambers were filled with either a nucleic acid (Examples 1-3) or red dye (Example 4) solution in 10 mM TrisHCl having a pH of 8.0 via a syringe pump at 40 μl/min. Pictures of the chambers were taken before and after filling and the movement of the solution in the chambers was video-taped during filling. Filling efficiencies were determined as set forth above (FEc) for the centered dried reagent. For the inlet-side dried reagent, the filling efficiency, FEi(%), was calculated based on a number of chambers in a microfluidic chip as FEi (%)=100*(WFi/Wid), where Wid is the number of chambers having dried reagent positioned at an inlet side of the chamber (e.g., proximate the inlet channel) per microfluidic chip and WFi is the number of chambers with no bubble formation after filling the chambers having inlet-side dried reagent.

FIGS. 10-16, 18, 19 and 21 show various images of the microfluidic chips taken during and after testing (e.g., filling of the microfluidic chips). In calculating the filling efficiencies for the inlet side and centered positioning of the dried reagents (i.e., as appearing in FIGS. 10A, 10B, 12A, 12B, 14A, and 14B), only 20 chambers per chip were used for the calculations. Four chambers in the column farthest to the right of the inlet of the microchip (e.g., as shown in FIG. 1) were excluded from the calculations since those chambers demonstrated a high frequency of bubble formation in the absence of dried reagent. It is believed that the high frequency of bubble formation observed in those chambers in the absence of dried reagent may be due to the inlet and outlet channel configurations differing from those for the 20 chambers shown. FIG. 1 schematically depicts the four chambers 20 contained in the far right column of the figure and their respective inlet and outlet channel configurations, as compared to the remaining columns of chambers.

Results of the various comparative studies are presented below.

### Example 1

### Filling of Chambers Having Centered Dried Reagent

135 nL of liquid reagent was dispensed at the center of the sample chambers of microfluidic chips by a liquid reagent dispenser and then dried (e.g., lyophilized). FIG. 10A is a photograph of a portion of a microfluidic chip 110 showing a plurality of chambers 120 having the dried reagent R (indicated by the white spots) positioned in the center of the chambers. As mentioned above, the 20 chambers shown in FIG. 10A were the chambers used in the calculation of the filling efficiency. The chips containing the centered dried reagent, as depicted in FIG. 10A, were then laminated as described above. The chips 110 also included a hydrophobic membrane 135 for ventilation (shown via the white strip in FIG. 10A), as described above. The main fluid channel 126 was connected to a syringe pump at a left-hand, top side of the channel 126 in FIG. 10A via an inlet (not shown) and the chambers 120 were filled via the main fluid channel 126 and inlet branch channels 122 with nucleic acid solution.

FIG. 10B shows a snapshot of the portion of the chip of FIG. 10A after filling of the chambers 120 with the nucleic acid solution. As can be seen in FIG. 10B, some of the chambers 120 contain bubbles B trapped within them after they have been filled (note that not all of the bubbles in FIG. 10B are labeled). After filling, the chambers 120 with no bubble formation (based on the 20 chambers included for each chip tested) were counted to determine the value of WFc and the filling efficiency, FEc, was calculated as set forth above, with Wcd being 20. Based on 11 chips tested, the average filling efficiency per chip, FEc, for chambers containing centered dried reagent was calculated as 47.6%±12.3.

Movement of the sample meniscus in the chambers was additionally video-taped. FIG. 11 shows various snapshots in time of chambers containing centered dried reagent being filled with sample. In particular, FIG. 11A shows various snapshots of filling a chamber 120 having centered dried reagent R in which no bubble entrapment occurred, while FIG. 11B shows snapshots of filling of another chamber 120 having centered dried reagent R in which bubble entrapment did occur. (Note that the same chamber is shown for each of the snapshots in FIG. 11A and the same chamber, different from that in FIG. 11A, is shown for each of the snapshots in FIG. 11B.) In each of the photos at the left-most position in FIGS. 11A and 11B, fluid was supplied via the channel 122 disposed toward the bottom left corner of each chamber 120. The meniscus M formed by the traveling sample fluid where observable is labeled in FIGS. 11A and 11B, and the bubble B trapped in the filled chamber 120 is labeled in FIG. 11B. In general, the movement of the meniscus M in the chambers 120 containing centered dried reagent was observed to be similar to the movement of the meniscus M in chambers containing no dried reagent.

### Example 2

### Filling of Chambers Having Dried Reagent Positioned at an Inlet Side

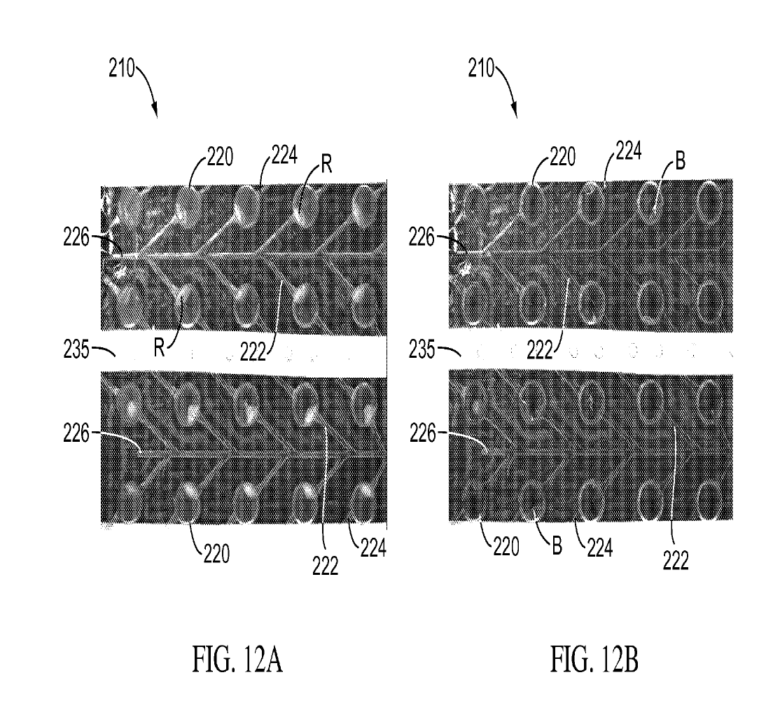

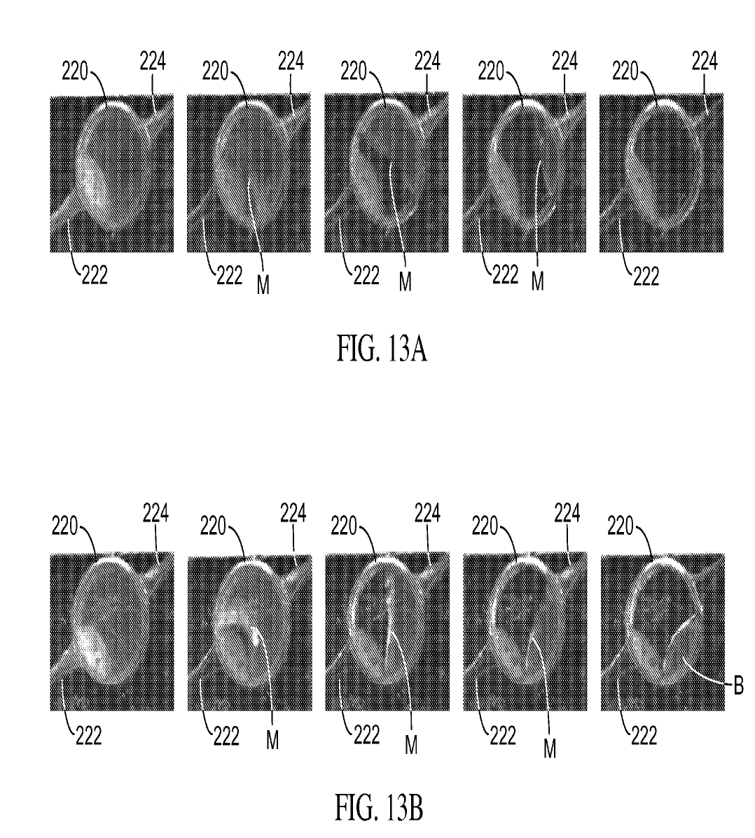

135 nL of liquid reagent was dispensed toward an inlet side (e.g., proximate the inlet channel) of all but two of the chambers of microfluidic chips by a liquid reagent dispenser and then dried (i.e., lyophilized). The two chambers in which reagent was positioned toward an outlet side were chambers positioned in the farthest column to the right from the fluid inlet (as shown in FIG. 1 and not shown in FIGS. 12A and 12B) and the four chambers in that column were excluded from calculating the filling efficiency. FIG. 12A is a photograph of a portion of a microfluidic chip 210 showing a plurality of chambers 220 having the dried reagent R (indicated by the white spots) positioned at an inlet side of the chambers 220 proximate the inlet channel 222 of the chambers 220. The 20 chambers shown in FIG. 12A were the chambers used in the calculation of the filling efficiency. The chips containing the inlet side dried reagent, as depicted in FIG. 12A, were then laminated with double-sided PSA film, as described above. The chips 210 also included a hydrophobic membrane 235 (white strip in FIG. 12 for ventilation), as described above. The main fluid channel 226 was connected to a syringe pump at a left-hand, top portion of the channel 226 in FIG. 12A and the chambers 220 were filled via the main fluid channel 226 and inlet branch channels 222 with the nucleic acid solution.

FIG. 12B shows a snapshot of the portion of the chip of FIG. 12A after filling of the chambers 220 with the nucleic acid solution. As can be seen in FIG. 12B, some of the chambers 220 contain bubbles B trapped within them after they have been filled (note that not all of the bubbles are labeled in FIG. 12B). After filling, the chambers 220 with no bubble formation (based on the 20 chambers included for each chip tested) were counted to determine the value of WFi, and the filling efficiency, FEi, was calculated as set forth above, with Wid being 20. Based on 11 chips tested, the average filling efficiency per chip, FEi, for chambers containing inlet-side positioning of the dried reagent was calculated as 65.0%±9.6.

Movement of the meniscus in the chambers was additionally video-taped. FIG. 13 shows various snapshots in time during the filling of the chambers containing inlet side dried reagent. In particular, FIG. 13A shows various snapshots of filling of a chamber 220 having inlet side dried reagent R in which no bubble entrapment occurred, while FIG. 13B shows snapshots of filling of a chamber 220 having inlet side dried reagent R in which bubble entrapment did occur. In each of the photos at the left-most position in FIGS. 13A and 13B, sample fluid was supplied via the channel 222 disposed toward the bottom left corner of each chamber 220. The meniscus M formed by the traveling sample fluid, where observable, is labeled in FIGS. 13A and 13B, and the bubble B trapped in the filled chamber 220 is labeled in FIG. 13B.

For chambers having inlet side dried reagent, the so-positioned reagent tended to guide the sample (nucleic acid solution) to come into the chamber relatively symmetrically against the center line connecting the inlet and outlet channels 222 and 224 in FIG. 13A. Once both ends of the meniscus M started moving toward the outlet channel 224, for example, as depicted in the second snapshot from the left in FIG. 13A, no bubble formed as long as the rate of travel of the entire meniscus remained similar.

As the surface of the chambers 220 are substantially hydrophobic (e.g., due to the plastic material from which they are made), adding the dried reagent at the inlet side tended to make the chamber surface at that location “virtually” hydrophilic. In other words, the reagent at the inlet side tended to absorb the sample as it entered the chamber 220 and cause the initial meniscus propagation to be flat (e.g., uniformly approaching the outlet channel 224) at the inlet side. This tended also to assist in making further meniscus propagation substantially uniform.

### Example 3

### Filling of Chambers Having Dried Reagent Positioned at an Inlet Side

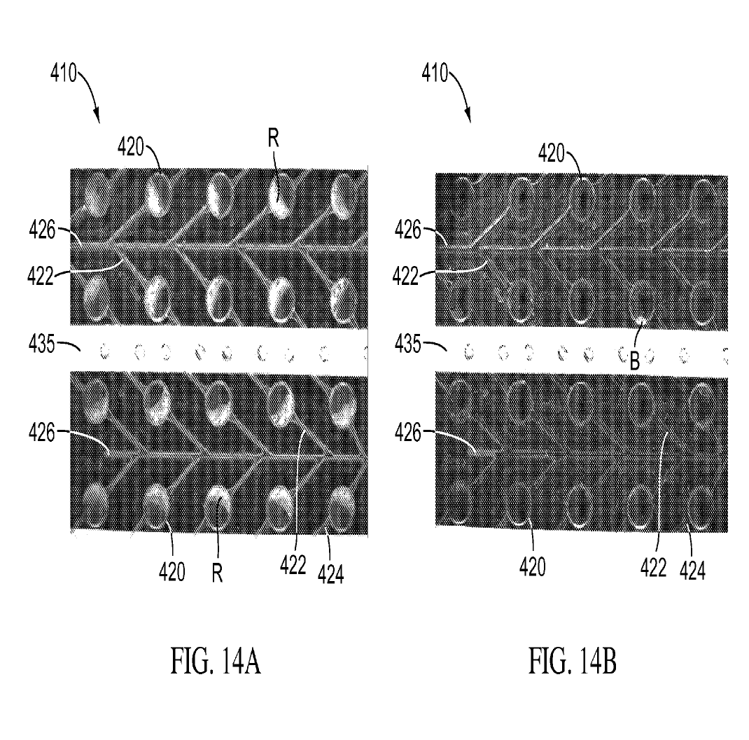

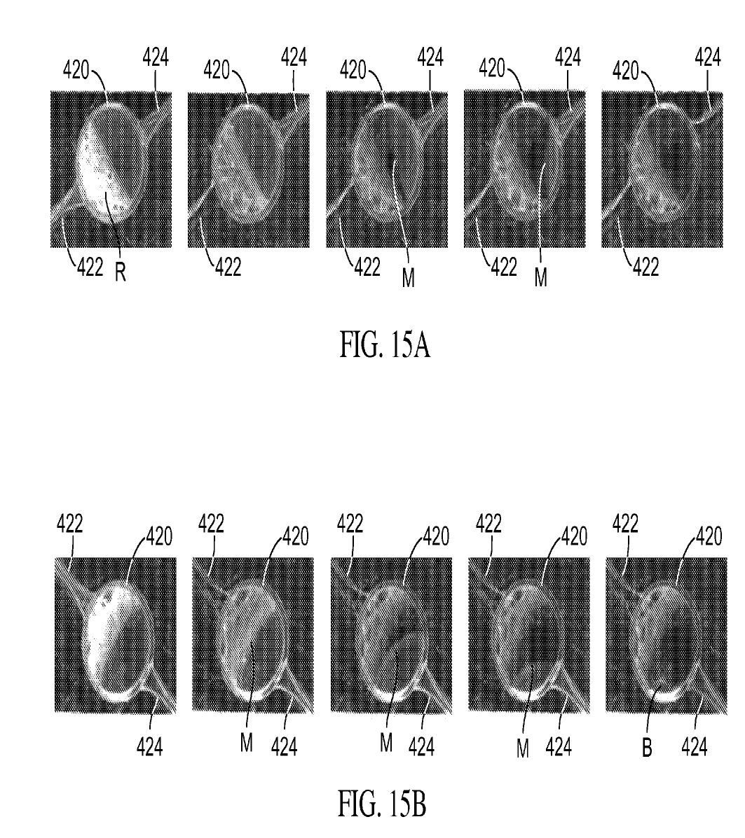

In an attempt to increase the filling efficiency of chambers containing inlet side dried reagent, tests were performed using a higher volume of liquid reagent dispensed on the inlet side of the chambers of microfluidic chips. In these tests, 260 nL of liquid reagent was dispensed toward an inlet side (e.g., proximate the inlet channel) of all but two of the chambers of microfluidic chips by a liquid reagent dispenser and then dried (i.e., lyophilized). The two chambers in which reagent was positioned toward an outlet side were chambers positioned in the farthest column to the right from the fluid inlet (as shown in FIG. 1 and not shown in FIGS. 14A and 14B) and the four chambers in that column were excluded from calculating filling efficiency. FIG. 14A is a photograph of a portion of a microfluidic chip 410 showing a plurality of chambers 420 having the dried reagent R (indicated by the white spots) positioned at an inlet side of the chambers 420 proximate the inlet channel 422 of the chambers 420. As with Examples 1 and 2, not all of the chambers of the chip 410 were used in calculating the filling efficiency, but rather only the 20 chambers shown in FIG. 14A. The chips containing the inlet side dried reagent, as depicted in FIG. 14A, were laminated with double-sided PSA film and included a hydrophobic membrane 435 (white strip shown in FIGS. 14A and 14B) for ventilation, as described above. The main fluid channel 426 was connected to a syringe pump at a left-hand, top side of the channel 426 in FIG. 14A and the chambers 420 were filled via the main fluid channel 426 and inlet branch channels 424 with a nucleic acid solution.

FIG. 14B shows a snapshot of the portion of the chip of FIG. 14A after filling of the chambers 420 with the nucleic acid solution. As can be seen in FIG. 14B, some of the chambers 420 contain bubbles B trapped within them after they have been filled (again note that not all of the bubbles are labeled in FIG. 14B). After filling, the chambers 420 with no bubble formation (based on the 20 chambers included for each chip tested) were counted to determine the value of WFi, and the filling efficiency, FEi, was calculated as set forth above, with Wid being 20. Based on 25 chips tested, the average filling efficiency per chip, FEi, for chambers containing 260 nL of inlet-side dried reagent was calculated as 95.0%±7.0.

Movement of the sample meniscus in the chambers also was video-taped. FIG. 15 shows various snapshots in time during the filling of the chambers containing 260 nL of inlet side dried reagent. In particular, FIG. 15A shows various snapshots of filling of a chamber 420 having inlet side dried reagent R like that described above in FIG. 14A in which no bubble entrapment occurred, while FIG. 15B shows snapshots of filling of a chamber 420 having inlet side dried reagent R in which bubble entrapment did occur. In each of the left-most photos in FIGS. 15A and 15B, sample fluid was supplied via the channel 422 disposed toward the bottom (FIG. 15A) or the top (FIG. 15B) left corner of each chamber 420. The meniscus M formed by the traveling sample fluid where observable is labeled in FIGS. 15A and 15B, and the bubble B trapped in the filled chamber 420 is labeled in FIG. 15B.

In Example 3, the increased amount of liquid reagent dispensed proximate the inlet side yielded dried reagent covering a greater area of the bottom surface of the chambers than in Example 2. The dried reagent in Example 3 thus guided the liquid sample approximately halfway to the outlet channel during filling of the chambers, thereby reducing the distance the sample had to travel to the outlet channel. In other words, the dried reagent acted as an absorption mechanism to absorb the liquid as it contacted the reagent in the chamber, making the chamber “virtually” hydrophilic at the location of the reagent, as discussed above. It is believed that bubble formation was reduced due to the shortened distance over which the sample is required to travel (e.g., without being guided by the reagent) through the chamber. In addition, as can be seen from the last snapshot on the right in FIG. 15B, bubbles that did form in the case of an increased amount of dried reagent present toward the inlet side of the chamber tended to be relatively small.

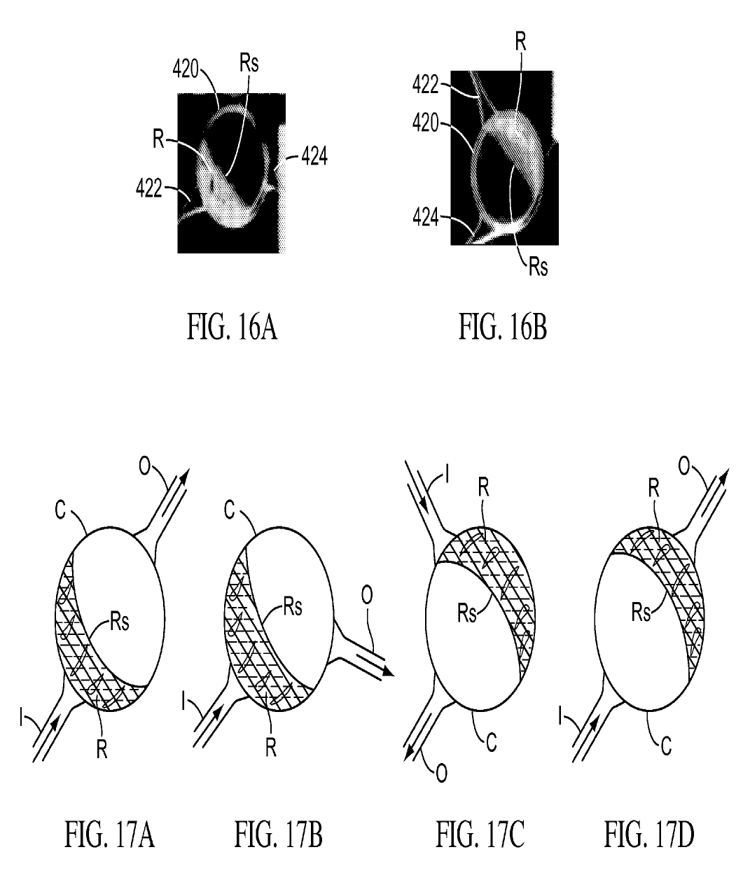

FIGS. 16A and 16B show a snapshot of two chambers 420 in Example 3 that were excluded from the filling efficiency calculation (e.g., two of the chambers from the column of four chambers positioned farthest to the right of the fluid inlet in the microfluidic chip exemplified in FIG. 1). In FIGS. 16A and 16B, the chambers 420 have an inlet channel 422 and an outlet channel 424 that are not 180 degrees apart from one another, as is the case with the chambers 420 depicted in FIGS. 14 and 15. In FIGS. 16A and 16B, dried reagent R is positioned proximate inlet channel 422 of the chambers 420. However, in FIG. 16A, the dried reagent surface Rs that faces toward a center of the chamber 420 faces in a direction that is nonperpendicular to the outlet channel 424. In FIG. 16B, the dried reagent surface Rs that faces toward a center of the chamber 420 is substantially perpendicular to the outlet channel 424. Schematic depictions of the positioning of the inlet and outlet channels and the dried reagent R in the chambers of FIGS. 16A and 16B can be seen in FIGS. 17B and 17C, respectively. In FIGS. 17B and 17C, the chambers are labeled C, the inlet channels are labeled I, the outlet channels are labeled O, the dried reagent is labeled R, and the dried reagent surface is labeled Rs.

Based on the filling of 10 microchips, the chambers having the inlet and outlet channel geometry and dried reagent positioning of FIG. 16A filled 50% of the time, while those having the channel configuration and reagent positioning of FIG. 16B filled 90% of the time. This observation indicates that the substantial perpendicularity of the dried reagent surface Rs to the outlet channel (e.g., the configuration of FIG. 16B and schematically depicted in FIG. 17C) may be a significant factor to filling chambers without bubble formation. In addition, based on the testing results for the examples above, positioning dried reagent at the inlet side of the chambers also is a significant factor to filling the chambers without bubble formation, particularly if the inlet and outlet channels are 180 degrees apart.

Thus, by positioning the dried reagent such that the surface of the reagent facing the center of the chamber is substantially perpendicular to the outlet channel, (e.g., as shown in FIGS. 16B, 17A, and 17C) the reagent may tend to guide the meniscus of the liquid sample in a desired manner so that the differing portions of the meniscus are substantially the same distance from the outlet channel. That is, because differing portions of the reagent surface facing the center of the chamber are approximately the same distance from the outlet channel, the meniscus, guided by the reagent, also has differing portions substantially the same distance from the outlet channel and tends to move through the chamber in this fashion. This tends to prevent one side of the meniscus from reaching the outlet channel before another side, so as to prevent bubble entrapment resulting from the blocking of the outlet channel by the sample. On the other hand, when one side of the dried reagent is closer to the outlet channel than the other, as shown FIG. 16A and FIG. 17 B, the one side of meniscus starting from the side of the dried reagent closer to the outlet channel may reach the outlet channel earlier than the other, again due to the reagent's tendency to guide (e.g., absorb) the sample as it enters the chamber, and block gas (e.g., air) from escaping. As explained previously, bubble formation may occur when one side of the meniscus reaches the outlet channel before the other side.

### Example 4

### Filling of Chambers Having Dried Reagent Positioned at an Outlet Side

To further determine the impact of the positioning of dried reagent within chambers of a microfluidic chip on bubble formation, an experiment was performed using dried reagent positioned at an outlet side of the chambers. In this experiment, 135 nL of liquid reagent was dispensed toward the outlet side (e.g., proximate the outlet channel) by a liquid reagent dispenser and then dried (i.e., lyophilized). FIG. 18A is a photograph of a portion of a microfluidic chip 810 showing a plurality of chambers 820 having the dried reagent R (indicated by the white spots) positioned at an outlet side of the chambers 820 proximate the outlet channel 822 of the chambers 820. The chip 810, as depicted in FIG. 18A, was laminated with a double-sided PSA film and included a hydrophobic membrane 835 (white strips shown in FIGS. 18A and 18B) for ventilation, as described above. The main fluid channel 826 was connected to a syringe pump at a left-hand, top portion of the channel 826 in FIG. 18A and the chambers 820 were filled via the main fluid channel 826 and inlet branch channels 824 with a red-dye solution in 10 mM Tris HCl having a pH of 8.0. In contrast to Examples 1-3, all 24 chambers 820 in the microfluidic chip 810 were used in the calculations to determine filling efficiency.

FIG. 18B shows a snapshot of the chip 810 of FIG. 18A after filling of the chambers 820 with the red dye solution. As can be seen in FIG. 18B, all of the chambers 820 contain bubbles B trapped within them after they being filled. Based on the single chip tested, therefore, the filling efficiency per chip having outlet side positioned dried reagent was calculated as 0%.

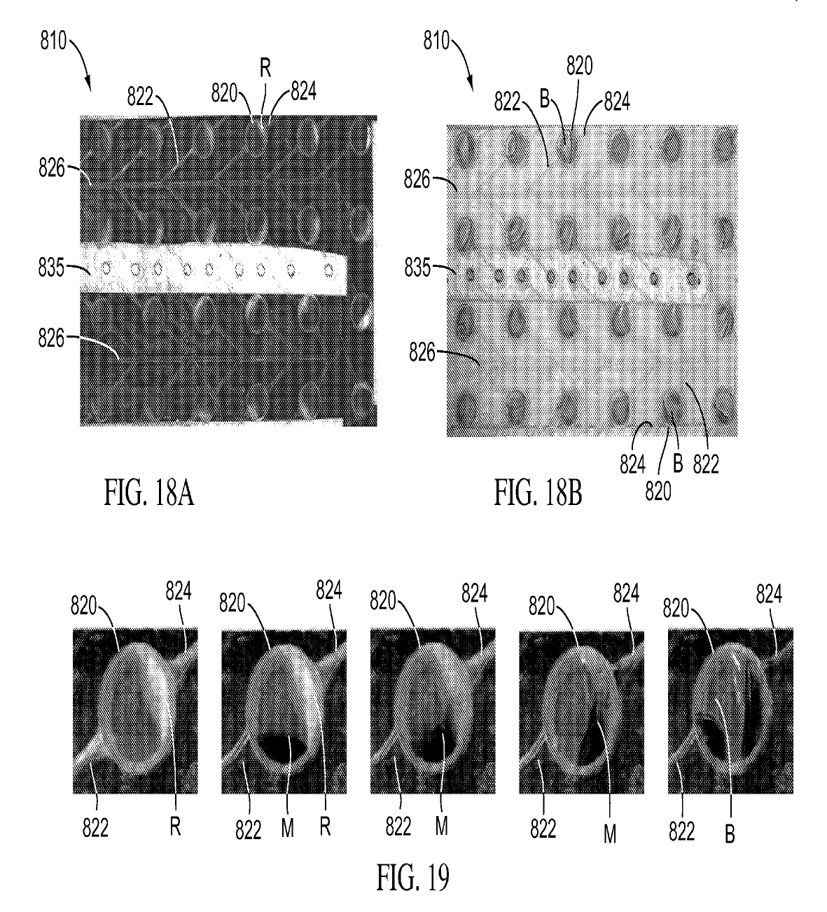

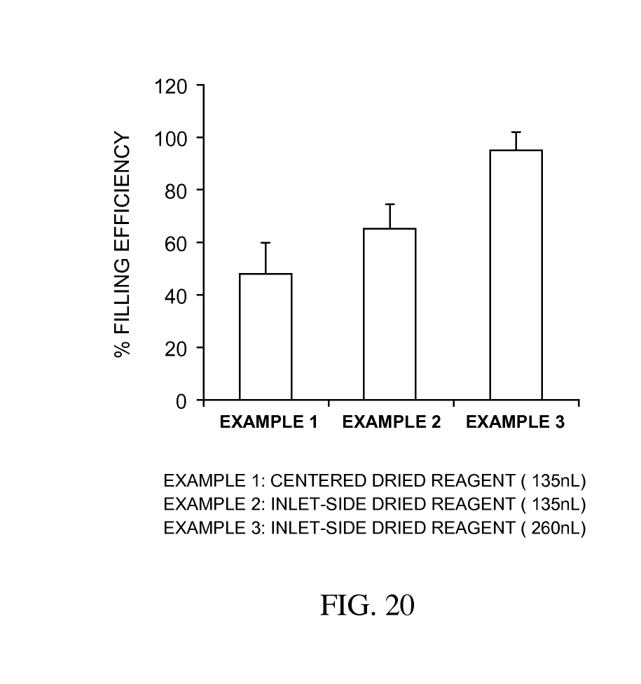

Movement of the sample meniscus in the chambers 820 also was video-taped. FIG. 19 shows various snapshots in time during the filling of the chambers 820 containing outlet side dried reagent. In the left-hand most snapshot of FIG. 19, sample solution was supplied via the inlet channel 822 disposed toward the bottom left corner of each chamber 820. Where observable, the meniscus M formed by the traveling sample solution and the bubble B trapped in the filled chamber are labeled in FIG. 19.

Positioning dried reagent at an outlet side of the chamber tends to bring a portion of the traveling sample meniscus that reaches the reagent first to the outlet channel before a portion of the meniscus that may lag behind. As described above, this may result in one portion of the meniscus reaching the outlet channel before the other side, thus blocking the outlet channel from displacing gas from the chamber and causing a bubble to become trapped in the chamber.

To summarize the results of the various examples presented above, it was determined that the average filling efficiency for chambers in a microfluidic chip in which 135 nL of liquid reagent dispensed and dried at a center position within the chambers was 47.6%±12.3 per chip, and was 65.0%±9.6 per chip for chambers having the same amount of liquid reagent dispensed and dried at an inlet side position (e.g., the chamber/reagent configuration substantially as depicted schematically in FIG. 17A). The average filling efficiency per chip for chambers in a microfluidic chip in which 260 nL of liquid reagent was dispensed and dried at an inlet side position within the chambers (e.g., for the chamber/reagent configuration substantially as depicted schematically in FIG. 17A) was 95.0%±7.0. And the filling efficiency for chambers in a microfluidic chip in which 135 nL of liquid reagent was dispensed and dried at an outlet side of the chambers (e.g., the chamber/reagent configuration substantially as depicted in FIG. 17D) was 0%. In other words, the outlet side positioned dried reagent resulted in bubble entrapment in all chambers.

FIG. 20 is a bar chart depicting the filling efficiency results of Examples 1-3 above, with the number of chips used in calculating the average filling efficiency per chip shown in each bar in the chart.

As can be observed from the results discussed above, the inlet side positioning of the dried reagent led to an increase in filling efficiency, and a greater amount of dried reagent (e.g., 260 nL vs. 135 nL) also significantly increased the filling efficiency. Based on the filling efficiency test results and observations of the solution filling the chambers, it is believed that dried reagent positioned at the inlet side guides the meniscus to move substantially perpendicularly to the outlet channel and shortens the distance the meniscus has to move within the chamber (e.g., a hydrophobic chamber of a microfluidic chip) to reach the outlet (i.e., due to the reagent absorbing the sample fluid as it travels within the chamber), which assists in preventing bubble formation and entrapment. In other words, it is believed that, although the chambers of the microfluidic chips are substantially hydrophobic, the dried reagent positioned at the inlet side of the chip tends to increase the hydrophilicity of the chip, which makes the chambers “virtually” hydrophilic in the region where the reagent is positioned. This in turn guides the sample through the chamber toward the outlet channel in a way that facilitates the meniscus's movement in a substantially uniform manner such that all portions of the meniscus reach the outlet channel at substantially the same time.

Further, as was discussed in Example 3, when dried reagent was deposited at the inlet side but not perpendicular to the outlet (e.g., as depicted in FIG. 16A and schematically in FIG. 17B), 50% of the chambers contained bubbles after filling. When the dried reagent was positioned perpendicularly to the outlet and proximate the inlet side in the position shown in FIG. 16B and schematically in FIG. 17C, 10% of the chambers formed bubbles after the fill. Based on the above, therefore, it was found that positioning dried reagent at or proximate an inlet side of the microfluidic chamber and facing in a direction substantially perpendicular to the outlet channel, for example, as schematically depicted in FIGS. 17A and 17C, facilitates moving the meniscus through the chamber in a substantially uniform manner such that bubble formation and entrapment is prevented when filling the chamber, as discussed above.

FIG. 21 shows snapshots of the filling of chambers 420 having an advantageous positioning of a dried reagent R within the chambers 420 of a microfluidic card. In particular, FIG. 21 shows snapshots of filling a chamber 420 having a reagent/channel configuration as shown in FIG. 16B and schematically in FIG. 17C, with the inlet and outlet channels not aligned with each other (i.e., separated by less than) 180° and reagent positioned proximate the inlet with the surface facing the center of the chamber 420 being substantially perpendicular to the outlet channel 424. In FIG. 21, the sample solution is introduced via the inlet channel 422. The progression of the meniscus M toward the outlet channel 424 of each the chamber 420 is shown in the snapshots. As can be seen by the last snapshot in the series presented in FIG. 21, no bubbles were entrapped after filling the chamber 420.

With reference now to FIG. 23, another exemplary arrangement of sample chambers 2320 of a microfluidic device is illustrated. As shown, each sample chamber 2320 may be in flow communication with an inlet branch channel 2322 and an outlet branch channel 2324. The inlet branch channels 2322 may in turn be in flow communication with main fluid channel portions 2326, 2327, and 2328, which may or may not be in flow communication with each other. Each sample chamber 2320 includes dried reagent R positioned toward an inlet side of the chamber 2320 proximate the opening of the inlet channel 2322. To facilitate the positioning (e.g., spotting) of the dried reagent R in each sample chamber 2320, for example, via a multi-tip spotter, the exemplary embodiment of FIG. 23 includes a substantially uniform distance (e.g., pitch) in all directions between the locations in each sample chamber 2320 at which it is desired to position the reagent R. That is, as shown in FIG. 23, the horizontal distance, X, between each dried reagent position and the vertical distance, Y, are the same over the entire array of sample chambers 2320. In an exemplary aspect, the distance X and Y may be about 4.5 mm. Providing a substantially uniform pitch in all directions (e.g., both the horizontal and vertical directions shown in FIG. 23), may facilitate desired placement of the dried reagent in all of the chambers 2320 of the microfluidic device, assuming all of the chambers 2320 are configured substantially the same with respect to their inlet channel and outlet channel orientations, as shown, for example, in FIG. 23. In other words, in the exemplary embodiment of FIG. 23, the inlet channel 2322 and outlet channel 2324 for each chamber 2320 of the array are positioned 180 degrees apart. Further, the inlet channel 2322 and outlet channel 2324 join each chamber 2320 at the same relative locations, for example, approximately at a bottom and a top position, as depicted in FIG. 23. Filling sample chambers 2320 of substantially uniform pitch using a multi-tip spotter that has spotting tips placed equidistant from each other may facilitate proper, automated positioning of the spotter at the desired location relative to the sample chambers 2320 to promote desired positioning of the dried reagent R. Although it may be desirable to have X and Y equal to each other, according to various exemplary embodiments, the values for X and Y may differ. In any case, according to various embodiments, the value of X and/or Y may be less than or equal to about 9 mm, for example, about 4.5 mm, or, for example, about 2.25 mm, or, for example, about 1.1 mm, etc.

As mentioned above, the exemplary embodiment of FIG. 23 also includes inlet channels 2322 and outlet channels 2324 situated approximately 180 degrees apart from each for each sample chamber 2320. As has been discussed, separating the inlet channel 2322 and outlet channel 2324 by 180 degrees may permit the sample meniscus to move within the chamber 2320 such that substantially the entire sample front reaches the outlet channel 2324 at the same time, thereby minimizing the potential to entrap a bubble in the chamber 2320. Further, with each sample chamber 2320 having the inlet and outlet channels 2322 and 2324 separated by 180 degrees, spotting of dried reagent can occur within each chamber 2320 at substantially the same location relative to both the inlet and outlet channels 2322 and 2324.

As discussed above, controlling the position of dried reagent within the sample chambers may substantially reduce or prevent bubble entrapment in the chamber during filling. For example, it may be desirable to position the dried reagent proximate an inlet side of the sample chambers. To position dried reagent in the sample chambers, reagent in liquid form may be dispensed (e.g., spotted) in the chamber, for example, toward the inlet side of the chamber, and dried (e.g., lyophilized). Relatively tight tolerances may be required to position dispensing devices (e.g., dispensing tips) at the appropriate location relative to the sample chambers to place the reagent at a desired location within the sample chambers. Also, liquid reagent may have a tendency to spread from its desired location within the sample chamber while it is drying. In cases where the liquid reagent is dispensed proximate the inlet side of the chamber, the reagent may tend to spread toward the outlet channel of the chamber, for example.

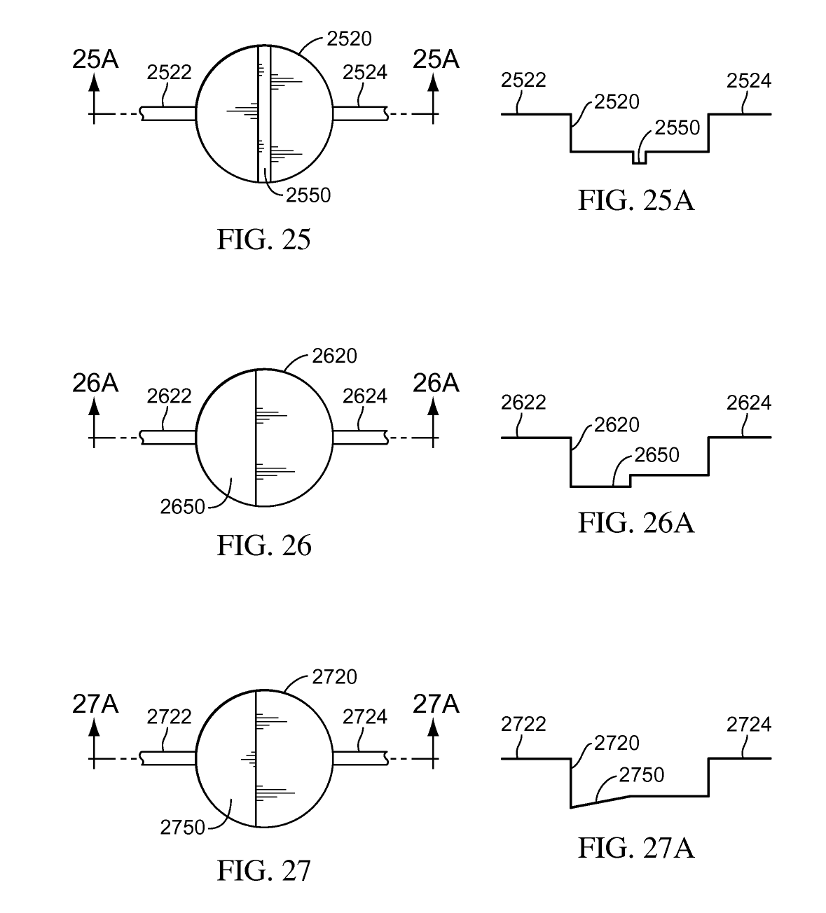

The exemplary embodiment of the sample chamber 20a of FIG. 3A, discussed above, included a ridge 36a positioned substantially at the center of the sample chamber 20a between the inlet channel 22a and the outlet channel 24a. As described with reference to the embodiment of FIG. 3A, the ridge 36a may assist in controlling the position of dried reagent in a chamber by stopping the spread of the liquid reagent past the ridge 36a if the reagent is deposited (e.g., spotted) toward an inlet side of the chamber 20a (e.g., proximate the inlet channel 22a). FIGS. 25-30 depict various other exemplary embodiments of sample chambers that are configured to control the positioning of a dried reagent within the sample chamber. By way of example, FIGS. 25-30 depict various features (e.g., modifications) included in a sample chamber to substantially hinder or prevent liquid reagent spotted toward an inlet side of the sample chamber from spreading in an undesired manner from the inlet side toward the outlet side as the reagent dried.

With reference to FIGS. 25 and 25A, according to various embodiments, the sample chamber 2520 may be provided with a small groove 2550 located substantially in the center of the chamber 2520 between the inlet channel 2422 and outlet channel 2524. The groove 2550 may extend substantially across the chamber 2520 in a direction substantially perpendicular to the inlet channel 2522 and outlet channel 2524, as shown in FIG. 25 (note that the A series of figures for FIGS. 25-30 represent the cross-section of each figure taken along the cross-section line shown in each figure.) The groove 2550 may be configured so as to trap liquid reagent that is spotted in the chamber 2520 toward the inlet channel 2522 and to prevent the liquid reagent from spreading past the groove 2550 in a direction toward the outlet channel 2524 as it dries. Although the groove 2550 depicted in FIGS. 25 and 25A has a substantially square profile, the groove 2550 may have any configuration, including, but not limited to, for example, triangular, circular, elliptical, etc. Also, instead of a groove, a ridge like that of FIG. 3A may be provided and have any configuration in accordance with the teachings herein.

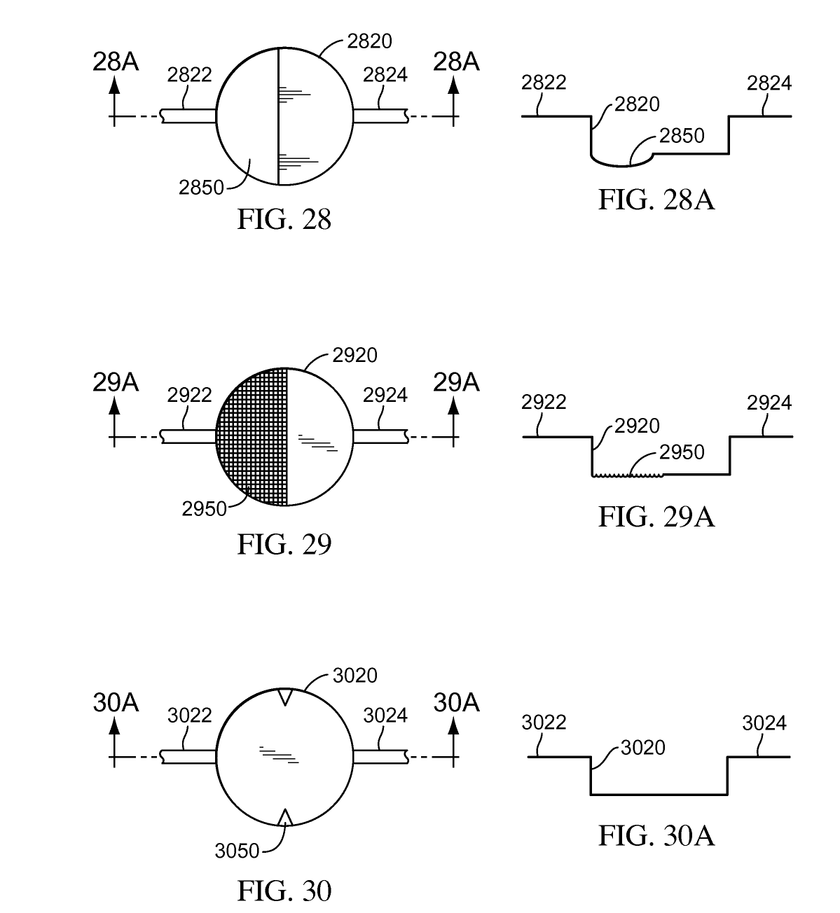

FIGS. 26-28A illustrate other exemplary embodiments of sample chambers that include physical modifications that may assist in controlling the spreading of dispensed liquid reagent in the sample chambers so as to control the location of dried reagent in the chambers. In each of the embodiments of FIGS. 26-28, the chambers 2620, 2720, and 2820 are provided with a small pocket (e.g., well) 2650, 2750, and 2850 configured to trap the dispensed liquid reagent and keep it from spreading. In the embodiments of FIGS. 26-28, the pockets 2650, 2750, and 2850 are formed by providing a deeper region of the chamber 2620, 2720, and 2820 between the inlet channel and substantially the center of the chamber 2620, 2720, and 2820. The pockets 2650, 2750, and 2850 may stop liquid reagent dispensed toward the inlet side of the chambers 2620, 2720, and 2820 from spreading away from the inlet side past the edge of the pockets 2650, 2750, and 2850 near the center of the chambers 2620, 2720, and 2820. As shown in FIGS. 26A, 27A, and 28A, the pockets 2620, 2720, and 2820 may have various configurations, including, but not limited to, for example, a substantially square profile (FIG. 26A), a substantially triangular profile (FIG. 27A), and a substantially round profile (FIG. 28A). Other profiles may also be suitable and are considered within the scope of the invention.

According yet further exemplary embodiments, a surface portion of the sample chamber may be modified so as to prevent the liquid reagent from spreading to undesirable locations within the chamber as it dries. FIGS. 29 and 29A depict an exemplary embodiment of a sample chamber 2920 that includes a roughened (e.g., textured) surface portion 2950 on a bottom surface of the sample chamber 2920. The roughened surface portion 2950 may cover approximately half of the sample chamber bottom surface from the inlet channel 2922 to substantially the center of the chamber 2920. Such texturing on the bottom surface portion 2950 of the sample chamber 2920 may substantially prevent a dispensed liquid reagent deposited proximate the inlet channel 2922 from spreading past the edge of the roughened surface portion 2950 at the center of the chamber 2920 and toward the outlet channel 2924. Providing the roughened and/or textured surface portion 2950 may act to increase the hydrophilicity of the surface portion 2950. Instead of texturing, other surface modifications that increase the hydrophilicity of the surface portion 2950 may be used to substantially prevent dispensed liquid reagent from spreading past the surface portion 2950 as it dries.

In the exemplary embodiments of FIGS. 25-29, bottom surface portions of the sample chambers include modifications configured to prevent dispensed liquid reagent from spreading past substantially the center of the chamber toward the outlet channel. In accordance with various embodiments, such modifications also may be provided on lateral surface portions of the sample chambers. FIGS. 30 and 30A depict an exemplary embodiment of a sample chamber 3020 that includes small protrusions 3050 extending from a lateral surface portion of the chamber 3020 toward a center of the chamber 3020. The small protrusions 3050 may be located substantially at the center of the chamber 3020 between the inlet channel 3022 and the outlet channel 3024 so as to prevent liquid reagent dispensed proximate the inlet channel 3022 from spreading in the chamber 3020 past the protrusions 3050 toward the outlet channel 3024. The protrusions 3050 may extend from approximately the bottom of the chamber 3020 and have a height ranging from about half the height of the chamber 3020 to about the full height of the chamber 3020. The protrusions 3050 in FIG. 30 have a substantially triangular cross-section, however, protrusions having other cross-sections may be used. Also, in lieu of a protrusion, an indentation (e.g., groove) may be provided in the lateral surface portion.

The various mechanisms described above and in accordance with exemplary aspects of the invention may provide enhanced control over the movement of the meniscus of a sample loading a sample-containment portion within a microfluidic device. Moreover, the various chamber modifications disclosed herein may facilitate the manufacturing of a microfluidic device that is configured to reduce or prevent the entrapment of gas bubbles within at least some of the sample-containment portions (e.g., chambers) of the device. In particular, since the various chamber features described herein may be manufactured or included as part of the microfluidic device on a macroscopic level, that is, as opposed to, for example, attempting to control (e.g., decrease) the surface roughness on a microscopic level, and/or chemically altering the chamber, providing such features to control the movement of the meniscus may be less complex and less costly. Further, at least some of the features described herein may be relatively insensitive to the wettability of the surface of the sample-containment portion and also relatively insensitive to contamination of the sample-containment portion, thereby providing control over the movement of the meniscus regardless of conditions which might be present within the sample-containment portion.

It should also be understood to those having skill in the art that the various exemplary embodiments described herein may be used individually or in combination with each other. Further, the various physical modifications described herein may be used in combination with surface treatments, washes, and other conventional techniques used for treating microfluidic devices.

Moreover, the techniques and devices described herein are applicable to any microfluidic device where an empty chamber, for example, a single chamber, is filled with liquid through an inlet and where the air displaced by the liquid is forced out of the chamber through an outlet. As such, the various devices and techniques described herein may be applicable to microfluidic device configurations other than those shown and described in the exemplary embodiments discussed above. By way of example, a microfluidic device may include a plurality of sample chambers that are serially connected such that the outlet of one chamber is the inlet of the next one. Further, a device in accordance with the teaching herein may include a combination of chambers connected in parallel and chambers connected in series. The present teachings for substantially hindering or preventing bubble entrapment are applicable to a variety of device configuration, including any of those mentioned above.

Although many of the embodiments discussed herein include microfluidic devices used in biological testing applications, it should be understood that various methods and devices in accordance with exemplary aspects may be applicable in a variety of other settings that require filling of microfluidic devices and for which the prevention or substantial hindering of bubble formation may be desirable. For example, it is envisioned that various exemplary embodiments may be useful in settings, such as, for example, drug delivery devices, inkjet applications, and other applications in which it is desirable to prevent the entrapment of air bubbles. Thus, the description of techniques, devices, and methods for substantially hindering or preventing bubble entrapment, as described herein, should be understood as exemplary and not limiting.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

It is noted that, as used in this specification and the appended claims, the singular forms “a,” “an,” and “the,” include plural referents unless expressly and unequivocally limited to one referent. Thus, for example, reference to “a reagent” includes two or more different reagents. As used herein, the term “include” and its grammatical variants are intended to be non-limiting, such that recitation of items in a list is not to the exclusion of other like items that can be substituted or added to the listed items.

It will be apparent to those skilled in the art that various modifications and variations can be made to the sample preparation device and method of the present disclosure without departing from the scope its teachings. Other embodiments of the disclosure will be apparent to those skilled in the art from consideration of the specification and practice of the teachings disclosed herein. It is intended that the specification and examples be considered as exemplary only.

1. A microfluidic device, comprising:
a sample distribution network comprising:
a plurality of sample chambers configured to be loaded with biological sample for biological testing of the biological sample while in the sample chambers,
a plurality of inlet channels, each inlet channel being in flow communication with and configured to flow biological sample to a respective sample chamber, and
a plurality of outlet channels, each outlet channel being in flow communication with and configured to flow biological sample from a respective sample chamber,
wherein at least some of the sample chambers comprise a dried reagent disposed within the at least some sample chambers proximate the inlet channels in flow communication with the at least some sample chambers.

2. The device of claim 1, wherein the dried reagent is positioned within the at least some sample chambers so as to control bubble formation within the at least some sample chambers.

3. The device of claim 2, wherein the at least some sample chambers are configured to control the position of the dried reagent proximate the inlet channels.

4. The device of claim 1, wherein the dried reagent is positioned in the at least some sample chambers such that a surface defined by the dried reagent and facing substantially toward a center of the at least some chambers extends substantially perpendicular to a longitudinal axis of the outlet channels in flow communication with the at least some sample chambers.

5. The device of claim 1, wherein the dried reagent increases the hydrophilicity of a portion of the at least some sample chambers on which the dried reagent is positioned in comparison to another portion of the at least some sample chambers.

6. The device of claim 1, wherein a pitch between locations of dried reagent in adjacent sample chambers is substantially the same.

7. The device of claim 1, wherein the at least some sample chambers are configured to control a position of the dried reagent within the at least some sample chambers.

8. The device of claim 7, wherein the at least some sample chambers comprise one of a physical modification and a surface modification configured to control the position of the dried reagent within the at least some sample chambers.

9. The device of claim 7, wherein the at least some sample chambers are configured to control the position of the dried reagent such that the dried reagent is not positioned in a region of the at least some chambers between approximately a center of the at least some chambers and the outlet channels in flow communication with the at least some chambers.

10. The device of claim 7, wherein each of the at least some sample chambers comprises one of a protrusion, a groove, a ridge, a region having a greater depth than other regions of each of the at least some sample chambers, a roughened surface portion, and a surface portion having greater hydrophilicity than other surface portions of each of the at least some sample chambers.

11. The device of claim 1, wherein the at least some sample chambers are configured to substantially prevent liquid reagent dispensed in the at least some sample chambers from spreading past a predetermined position as the liquid reagent dries.

12. The device of claim 1, wherein each of the plurality of sample chambers comprises a dried reagent disposed within each sample chamber proximate the inlet channels in flow communication with the at least some sample chambers.

13. A method of filling a microfluidic device, the method comprising:
supplying the microfluidic device with a biological sample, the microfluidic device comprising:
a plurality of sample chambers,
a plurality of inlet channels, each inlet channel being in flow communication with and configured to flow biological sample to a respective sample chamber, and
a plurality of outlet channels, each outlet channel being in flow communication with and configured to flow biological sample from a respective sample chamber,
wherein a dried reagent is positioned within at least some of the sample chambers proximate the inlet channels in flow communication with the at least some sample chambers; and loading the sample chambers with the biological sample.

14. The method of claim 13, further comprising controlling bubble formation within the at least some sample chambers via the dried reagent during loading.

15. The method of claim 13, further comprising controlling the position of the dried reagent within the at least some sample chambers.

16. The method of claim 15, wherein controlling the position of the dried reagent comprises controlling the position via one of a physical modification and a surface modification of the at least some sample chambers.

17. The method of claim 15, wherein controlling the position of the dried reagent comprises controlling the position of the dried reagent such that the dried reagent is not positioned in a region of the at least some chambers between approximately a center of the at least some chambers and the outlet channels in flow communication with the at least some chambers.

18. The method of claim 15, wherein controlling the position of the dried reagent comprises controlling the position of the dried reagent via one of a protrusion, a groove, a ridge, a region having a greater depth than other regions of each of the at least some sample chambers, a roughened surface portion, and a surface portion having greater hydrophilicity than other surface portions of each of the at least some sample chambers.

19. The method of claim 15, wherein controlling the position of the dried reagent comprises substantially preventing liquid reagent dispensed in the at least some sample chambers from spreading past a predetermined position as the liquid reagent dries.

20. The method of claim 15, wherein controlling the position of the dried reagent comprises providing a substantially uniform pitch between locations of adjacent sample chambers at which dried reagent is to be positioned.
